(19) United States
(12) Patent Application Publication
(10) Pub. No.: US 2026/0295267 A1
(43) Pub. Date: Oct. 1, 2026

(54) CLOSED-LOOP STIMULATION BASED ON MULTIPLE SENSING MODALITIES

(71) Applicant: Medtronic, Inc., Minneapolis, MN (US)

(72) Inventors: Benjamin P. Isaacson, Mahtomedi, MN (US); Leonid M. Litvak, Bet Shemesh (IL); David A. Dinsmoor, North Oaks, MN (US); David E. Linde, Bremerton, WA (US); David L. Carlson, Fridley, MN (US)

(21) Appl. No.: 19/479,568

(22) PCT Filed: Apr. 11, 2024
(86) PCT No.: PCT/IB2024/053552

§ 371 Date: Oct. 28, 2025

Related U.S. Application Data
(60) Provisional application No. 63/499,101, filed on Apr. 28, 2023.

(51) Int. Cl. A61N 1/36; A61B 5/11

(52) U.S. Cl. CPC A61N 1/36139; A61B 5/1118

(57) ABSTRACT

Devices, systems, and techniques are disclosed for managing electrical stimulation therapy and/or sensing of physiological signals such as brain signals. For example, a system control stimulation generation circuitry to deliver electrical stimulation therapy based on a first sensing modality configuration of the plurality of different sensing modality configurations and determine a change to at least one of a patient state or a therapy mode. The system may, responsive to determining the change, select a second sensing modality configuration of the plurality of different sensing modality configurations, and control the stimulation generation circuitry to deliver the electrical stimulation therapy based on the second sensing modality configuration instead of the first sensing modality configuration.

This Application claims priority from U.S. Provisional Patent Application 63/499,101, filed 28 Apr. 2023, the entire content of which is incorporated herein by reference.

### TECHNICAL FIELD

This disclosure generally relates to electrical stimulation therapy, and, more specifically, sensing modalities used for adjusting the electrical stimulation therapy ..

### BACKGROUND

Medical devices may be external or implanted, and may be used to deliver electrical stimulation therapy to various tissue sites of a patient to treat a variety of symptoms or conditions such as chronic pain, tremor, Parkinson's disease, other movement disorders, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Hence, electrical stimulation may be used in different therapeutic applications, such as deep brain stimulation (DBS), spinal cord stimulation (SCS), pelvic stimulation, gastric stimulation, or peripheral nerve field stimulation (PNFS).

A clinician may select values for a number of programmable parameters in order to define the electrical stimulation therapy to be delivered by the implantable stimulator to a patient. For example, the clinician may select one or more electrodes for delivery of the stimulation, a polarity of each selected electrode, a voltage or current amplitude, a pulse width, and a pulse frequency as stimulation parameters. A set of parameters, such as a set including electrode combination, electrode polarity, voltage or current amplitude, pulse width and pulse rate, may be referred to as a program in the sense that they define the electrical stimulation therapy to be delivered to the patient.

### SUMMARY

In general, the disclosure describes devices, systems, and techniques for managing DBS therapy, which may include switching between one or more different sensing modalities for different situations, such as different patient conditions and/or different therapy modes. For any given set of circumstances (e.g., therapy mode, patient condition, etc.) the system may employ a specific sensing modality configuration. Each sensing modality configuration may specify one, two, three, or more different sensing modalities that are used as feedback to control the delivery of therapy, such as electrical stimulation therapy. Each sensing modality configuration may have the one or more sensing modalities in order to provide appropriate feedback for a specific therapy mode, sense physiological data when possible, and/or reduce power consumption when certain sensing modalities are not needed for feedback.

For different therapy modes, the respective sensing modality configurations may include respective different one or more sensing modalities that are capable of sensing during that therapy mode and/or for different patient conditions. Example sensing modalities may include biochemical sensing (e.g., detecting the presence of one or more neurotransmitters, medications, etc.), electrophysical sensing (e.g., evoked responses, local field potentials (LFP), etc.), activity sensing (e.g., accelerometer signals), and posture sensing (e.g., accelerometer or pressure signals). Each sensing modality may provide information more relevant to certain therapy modes or patient conditions. Different sensing modalities may also employed by the system for other purposes, such as confirming the accuracy of a sense event from another sensing modality or switching to the different sensing modality in response to detecting an error with a primary sensing modality.

In one example, a system includes a memory configured to store a plurality of different sensing modality configurations, each sensing modality configuration of the plurality of sensing modality configurations defining at least one sensing modality of a plurality of sensing modalities; processing circuitry configured to: control stimulation generation circuitry to deliver electrical stimulation therapy based on a first sensing modality configuration of the plurality of different sensing modality configurations; determine a change to at least one of a patient state or a therapy mode; responsive to determining the change, select a second sensing modality configuration of the plurality of different sensing modality configurations; and control the stimulation generation circuitry to deliver the electrical stimulation therapy based on the second sensing modality configuration instead of the first sensing modality configuration.

In another example, a method includes storing, by a memory, a plurality of different sensing modality configurations, each sensing modality configuration of the plurality of sensing modality configurations defining at least one sensing modality of a plurality of sensing modalities; controlling, by processing circuitry, stimulation generation circuitry to deliver electrical stimulation therapy based on a first sensing modality configuration of the plurality of different sensing modality configurations; determining, by processing circuitry, a change to at least one of a patient state or a therapy mode; responsive to determining the change, selecting, by processing circuitry, a second sensing modality configuration of the plurality of different sensing modality configurations; and controlling, by processing circuitry, the stimulation generation circuitry to deliver the electrical stimulation therapy based on the second sensing modality configuration instead of the first sensing modality configuration.

In another example, a non-transitory computer-readable medium includes instructions that, when executed, control processing circuitry to store, in a memory, a plurality of different sensing modality configurations, each sensing modality configuration of the plurality of sensing modality configurations defining at least one sensing modality of a plurality of sensing modalities; control stimulation generation circuitry to deliver electrical stimulation therapy based on a first sensing modality configuration of the plurality of different sensing modality configurations; determine a change to at least one of a patient state or a therapy mode; responsive to determining the change, select a second sensing modality configuration of the plurality of different sensing modality configurations; and control the stimulation generation circuitry to deliver the electrical stimulation therapy based on the second sensing modality configuration instead of the first sensing modality configuration The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an implantable medical device (IMD) configured to deliver DBS to a patient according to an example of the techniques of the disclosure.

FIG. 2 is a block diagram of the example IMD of FIG. 1 for delivering DBS therapy according to an example of the techniques of the disclosure.

FIG. 3 is a block diagram of the external programmer of FIG. 1 for controlling delivery of DBS therapy according to an example of the techniques of the disclosure.

FIG. 4 is a flowchart illustrating an example technique for switching between different sensing modality configurations.

FIG. 5 is a flowchart illustrating an example technique for switching between different sensing modality configurations for different pulse frequencies of electrical stimulation.

FIG. 6 is a flowchart illustrating an example technique for switching between different sensing modality configurations in different posture states.

FIG. 7 is a flowchart illustrating an example technique for confirming sense events with a different sensing modality configuration.

### DETAILED DESCRIPTION

A patient may suffer from one or more symptoms treatable by electrical stimulation therapy. For example, a patient may suffer from brain disorder such as Parkinson's disease, Alzheimer's disease, or another type of movement disorder. Deep brain stimulation (DBS) may be an effective treatment to reduce the symptoms associated with such disorders. It may be time consuming for a clinician to manually determine appropriate stimulation parameters that define effective electrical stimulation therapy. Moreover, DBS is typically delivered continuously in an open loop fashion for the patient. Not only does this open loop delivery consume more battery power due to stimulation being delivered when not needed by the patient, but the system is not able to adjust stimulation parameters to provide more targeted therapy as the patient status changes, the condition of the patient changes over time or under certain conditions, or different therapies are delivered. In addition, even if a system can sense a patient condition for purposes of adjusting therapy, the sensing mode may not be appropriate for various therapy modes, patient events, or changes to the system.

To improve neurostimulation therapy and patient outcomes, closed loop stimulation therapy can be more customized, more responsive, more efficient, and/or easier to use. In order to achieve these closed-loop therapy benefits, the system may be configured to obtain an accurate estimation of patient state and/or disease state.

As described herein, various devices, systems, and techniques enable changing between different sets of sensing modalities that the system can use to control the delivery of stimulation therapy, such as DBS, spinal cord stimulation, etc. In the example of DBS, the system may operate in an adaptive DBS mode in which the system adjusts the value of one or more stimulation parameters that define electrical stimulation in order to maintain the brain signals above or below one or more respective thresholds. The system may receive user input specifying or adjusting any of these thresholds. The system may also receive user input specifying one or more sensing modalities to use for closed-loop control of stimulation for any therapy mode or different patient states. In this manner, multiple sensing modalities can be employed to perform this assessment in a strategic way that can yield the most powerful and efficacious system.

There may be certain changes in patient state or disease state that may benefit from a change to one or more sensing modalities when these patient state or disease states change. The optimization of neural sensing parameters, or other sensing parameters, for a given current sensing modality can improve behavior or patient treatment (e.g., a different biomarker that is acquired via a different sensing modality may provide better insight into the patient state, inform how to adjust therapy, etc.). However, using different sensing modalities altogether may further improve the ability of the system to obtain appropriate feedback information to adjust therapy over time. In this manner, a higher-level sensing modality optimization or priority scheme can be employed by the system to enable the system to acquire the most relevant biomarker (e.g., sense event or data from a specific sensing modality) at any given time.

Example types of sensing modalities may include biochemical sensing (e.g., detecting the presence of one or more neurotransmitters, medications, etc.), electrophysical sensing (e.g., evoked responses, local field potentials (LFP), electroencephalogram (EEG signals), electrocardiogram (ECG signals), electromyogram (EMG), etc.), activity sensing (e.g., accelerometer signals), and posture sensing (e.g., accelerometer or pressure signals). Each of these types of sensing modalities may include one or more different specific sensors or signal processing that provides specific information associated with a patient status or disease states. A sensing modality configuration described herein may specify one or more different sensing modalities that can be used together as feedback for controlling, or adjusting, stimulation therapy at a given time. In this manner, the system may switch between different sensing modality configurations in response to a switch to different therapy mode, different patient state, or any other aspect related to therapy has changed.

For different therapy modes, the respective sensing modality configurations may include respective different one or more sensing modalities that are capable of sensing during that therapy mode. For example, for electrical stimulation that includes pulse frequencies too high to sense evoked compound action potential (ECAP) signals between pulses, the system may utilize local field potential (LFP) sensing for feedback since LFP signals may still be detectable. The pulse frequency and pulse width may indicate the available time for sensing between each pulse. The system may switch to an ECAP signal sensing modality during therapy modes where the pulse frequency is below the threshold required to begin sensing the ECAP between pulses. This switching may continuously occur in response to cycling between different therapy modes over time, for example. In another example, the system may use an ECG sensing modality to sense electrocardiogram (ECG) signals (e.g., ECG sensing using LFP sensing circuitry) in response to accelerometer indications that the patient state may be sleeping. The system can then adjust stimulation therapy based on a sleep state at least partially detected using the ECG signals. Since the ECG signals may be detectable when the patient is still or sleeping, the accelerometer may control when the system should attempt to sense ECG signals for determining the sleep state of the patient which may inform therapy adjustments (e.g., reducing or during off stimulation therapy).

Different sensing modalities may also employed by the system for other purposes, such as confirming the accuracy of a sense event from another sensing modality. For example, the system may monitor accelerometer signals to confirm that LFP signals associated with neurological based movements are accurate to the movement of the patient (e.g., seizure, tremor, gait freeze, etc.). In another example, the system may be configured to switch to the different sensing modality in response to detecting an error with a primary sensing modality. This switching modalities can increase the fault tolerance of the system by having one or more backup sensing modalities that the system can use to continue controlling stimulation therapy over time.

These various features may provide advantages over other systems and improve system functionality and patient outcomes. The concepts described herein can enable a system to selectively, smartly, and efficiently switch between or fuse different sensing modalities to provide improved estimation of patient state or disease state that can inform therapy adjustments. In this manner, a stimulation therapy system can provide more accurate closed-loop control and/or more robust sensing over time. This sensing modality switching or combinations may improve therapeutic results for the patient by increasing therapeutic stimulation efficacy and reducing side effects via tracking patient conditions. The system may also correlate patient condition and events with sensed brain signals.

Although DBS therapy is described in various examples herein, these sensing modalities configurations and switched sensing can be applicable to any type of stimulation therapy or other therapy that may benefit from different sensing modalities. Example therapies may include spinal cord stimulation, pelvic floor stimulation (which may include sacral nerve modulation), incontinence therapy (e.g., sacral nerve stimulation and/or tibial nerve stimulation), peripheral nerve stimulation, occipital nerve stimulation, gastric stimulation, etc.

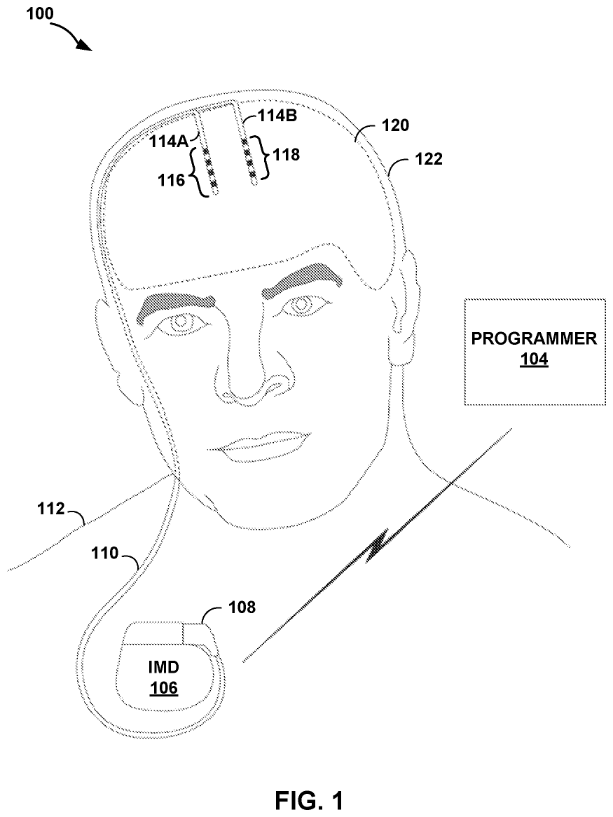

FIG. 1 is a conceptual diagram illustrating an example system 100 that includes an implantable medical device (IMD) 106 configured to deliver adaptive deep brain stimulation to a patient 112. DBS may be adaptive in the sense that IMD 106 may adjust, increase, or decrease the magnitude of one or more stimulation parameters that at least partially defines the DBS in response to sensed changes in one or more aspects related to the patient, such as patient activity or movement, a severity of one or more symptoms of a disease of the patient, a presence of one or more side effects due to the DBS, or one or more sensed signals of the patient, changed therapy modes, etc. For example, one or more sensed signals of the patient may be used as a control signal such that the IMD 106 correlates the magnitude of the one or more parameters of the electrical stimulation to the magnitude of the one or more sensed signals. According to the techniques of the disclosure, system 100, via IMD 106, delivers electrical stimulation therapy having one or more parameters, such as voltage or current amplitude, adjusted in response to a signal deviating from a range defined by a homeostatic window (e.g., a window defined by one or more thresholds for a brain signal, such as a lower threshold and upper threshold).

In other examples, the system delivers electrical stimulation therapy having the one or more parameters, such as voltage or current amplitude, adjusted in response to multiple signals, each signal deviating from a range defined by a respective homeostatic window. For example, the system may sense a first neurological signal, such as a signal within a Beta frequency band of the brain 120 of patient 112 within a first respective homeostatic window and a second neurological signal, such as a signal within a Gamma frequency band of the brain 120 of patient 112 within a second respective homeostatic window. In one example system, IMD 16 dynamically selects one of the first signal or the second signal for controlling adjustment of the one or more parameters based on a determination of which of the first signal or second signal most accurately corresponds to the severity of one or more symptoms of the patient. In another example system, IMD 106 adjusts the one or more parameters based on a ratio of the first signal to the second signal. In some examples, amplitudes of one or more frequencies in the Gamma frequency band increase with greater stimulation intensity such that higher Gamma frequency amplitudes may be associated with side effects. Conversely, amplitudes of one or more frequencies in the Beta frequency band decrease with greater stimulation intensity such that lower Gamma frequency amplitudes may be associated with side effects (e.g., dyskinesia). As described herein, IMD 106 may switch to different sensing modality configurations, such as different sensors, different types of electrical signals, etc., based on the patient condition and/or therapy mode that is used. Each sensing modality configuration may specify one or more different sensing modalities that can be used at the same time to control the current therapy being delivered.

In some examples, the medication taken by patient 112 is a medication for controlling one or more symptoms of Parkinson's disease, such as tremor or rigidity due to Parkinson's disease. Such medications include extended release forms of dopamine agonists, regular forms of dopamine agonists, controlled release forms of carbidopa/levodopa (CD/LD), regular forms of CD/LD, entacapone, rasagiline, selegiline, and amantadine. Typically, to set the upper threshold and lower threshold of the homeostatic window, the patient has been off medication, i.e., the upper and lower thresholds are set when the patient is not taking medication selected to reduce the symptoms. The patient may be considered to be not taking the medication when the patient, prior to the time the upper bound is set, has not taken the medication for at least approximately 72 hours for extended release forms of dopamine agonists, the patient has not taken the medication for at least approximately 24 hours for regular forms of dopamine agonists and controlled release forms of CD/LD, and the patient has not taken the medication for at least approximately 12 hours for regular forms of CD/LD, entacapone, rasagiline, selegiline, and amantadine. If only stimulation is suppressing brain signals (e.g., LFP signals), then the system can measure these brain signals for various values of stimulation parameters without outside inputs. Once the upper threshold and lower threshold is established, the system can identify when medication wears off because the brain signals will cross the lower or upper threshold. In response to identifying the brain signal crossing a threshold, the system may turn on electrical stimulation to bring back brain signal amplitudes back between the lower threshold and the upper threshold. Programmer 104 may enable the user to initially set the lower threshold and the upper threshold and make adjustments over time. Programmer 104 may also determine and display information regarding the amount of time stimulation amplitude is above, below, or between the thresholds. In some examples, a sensing modality configuration may include a biochemical signal generated by a biochemical sensor that may detect the presence of medication in the patient or a compound or molecule part of the patient's physiological response to the medication. However, IMD 106 may only operate the sensing modality configuration that includes the biochemical signal when medication may be taken in order preserve the functionality of the associated biochemical sensor that may degrade with use over time.

As described herein, “reducing” or “suppressing” the symptoms of the patient refer to alleviating, in whole or in part, the severity of one or more symptoms of the patient. In one example, a clinician makes a determination of the severity of one or more symptoms of Parkinson's disease of patient 112 with reference to the Unified Parkinson's Disease Rating Scale (UPDRS) or the Movement Disorder Society-Sponsored Revision of the Unified Parkinson's Disease Rating Scale (MDS-UPDRS). A discussion of the application of the MDS-UPDRS is provided by Movement Disorder Society-Sponsored Revision of the Unified Parkinson's Disease Rating Scale (MDS-UPDRS): Scale Presentation and Clinimetric Testing Results, C. Goetz et al, Movement Disorders, Vol. 23, No. 15, pp. 2129-2170 (2008), the content of which is incorporated herein in its entirety.

As described herein, a clinician can determine the upper threshold of the homeostatic window while the patient is not taking medication, and while, via IMD 106, electrical stimulation therapy is delivered to the brain 120 of patient 112. In one example, a clinician determines the point at which increasing the magnitude of one or more parameters defining the electrical stimulation therapy, such as voltage amplitude or current amplitude, begins to cause one or more side effects for the patient 112. For example, the clinician may gradually increase the magnitude of one or more parameters that the electrical stimulation therapy and determine the point at which further increase to the magnitude of one or more parameters defining the electrical stimulation therapy causes a perceptible side effect for patient 112. As described herein, IMD 106 may sense LFPs during this process and display the LFP signal and/or LFP signal magnitude that may correspond to the respective thresholds. In some examples, IMD 106 may use different or additional sensing modalities from LFPs in certain situations, such as during sleep for the patient.

As also described above, a clinician determines the lower threshold while the patient is off medication and while, via IMD 106, electrical stimulation therapy is delivered to the brain 120 of patient 112. In one example, a clinician determines the point at which decreasing the magnitude of one or more parameters defining the electrical stimulation therapy causes break-through of one or more symptoms of the patient 112. This break-through of symptoms may refer to re-emergence of at least some symptoms that were substantially suppressed up to the point of re-emergence due to the decrease in magnitude of the one or more electrical stimulation therapy parameters. For example, the clinician may gradually decrease the magnitude of one or more parameters defining the electrical stimulation therapy and determine the point at which the symptoms of Parkinson's disease in patient 112 emerge, as measured by sudden increase with respect to tremor or rigidity, in the score of patient 112 under the UPDRS or MDS-UPDRS. In another example, the clinician measures a physiological parameter of patient 112 correlated to one or more symptoms of the disease of patient 112 (e.g., wrist flexion of patient 112) and determines the point at which further decrease to the magnitude of one or more parameters defining the electrical stimulation therapy causes a sudden increase in the one or more symptoms of the disease of patient 112 (e.g., onset of lack of wrist flexion of patient 112).

At the magnitude of one or more parameters defining the electrical stimulation therapy at which further decrease to the magnitude of one or more parameters defining the electrical stimulation therapy causes a sudden increase in the one or more symptoms of the disease of patient 112, the clinician measures the magnitude of the signal of the patient 112 and sets this magnitude as the lower threshold of the homeostatic window. In some examples, the clinician may select a lower threshold of the homeostatic window to be a predetermined amount, e.g., 5% or 10%, higher than the magnitude at which the symptoms of the patient 112 first emerge during decrease in the magnitude of one or more electrical stimulation parameters to prevent emergence of the symptoms of the patient 112 during subsequent use.

In another example, the clinician sets the lower threshold by first ensuring that the patient is off medication for the one or more symptoms. In some examples, IMD 106 may operate in a sensing modality configuration that includes a biochemical signal that can indicate whether the patient is still being affected by any medication. In any case, the clinician delivers electrical stimulation having a value for the one or more parameters approximately equal to the upper threshold of the therapeutic window. In some examples, the clinician delivers electrical stimulation having a value for the one or more parameters slightly below the magnitude which induces side effects in the patient 112. Typically, this causes greater reduction of the one or more symptoms of the disease of the patient 112, and therefore greater reduction of the signal. At this magnitude of the one or more parameters, the clinician measures the magnitude of the signal of the patient 112 and sets, via external programmer 104, this magnitude as the lower threshold of the homeostatic window. In some examples, the clinician may select a value for the lower threshold of the homeostatic window to be a predetermined amount, e.g., 5% or 10%, higher than the magnitude at which the symptoms of the patient 112 emerge to prevent emergence of the symptoms of the patient 112 during subsequent use.

Additionally, in various examples of the techniques of the disclosure, the system monitors one or more signals of the patient based on the current sensing modality configuration employed by IMD 106. In one example, the sensing modality configuration may include sensing a signal that is a neurological signal (e.g., a bioelectrical signal) of a patient, such as a signal within a Beta frequency band or a Gamma frequency band of the brain of the patient. In yet a further example, the sensing modality configuration may include sensing a signal is a signal indicative of a physiological parameter of the patient, such as a severity of a symptom of the patient, a signal indicative of a posture of the patient, a signal indicative of a respiratory function of the patient, a signal indicative of an activity level of the patient, a signal indicative of a sleep state of the patient, or a signal indicative of other body functions such as cardiac functions via ECG, for example. For example, the monitored one or more signals of a sensing modality configuration may be a power of the respective Beta frequency band and/or Gamma frequency band. The system, via IMD 106, can be configured to deliver electrical stimulation to the patient, wherein one or more parameters defining the electrical stimulation are proportional to the magnitude of the monitored signal.

System 100 may be configured to treat a patient condition, such as a movement disorder, neurodegenerative impairment, a mood disorder, or a seizure disorder of patient 112. Patient 112 ordinarily is a human patient. In some cases, however, therapy system 100 may be applied to other mammalian or non-mammalian, non-human patients. While movement disorders and neurodegenerative impairment are primarily referred to herein, in other examples, therapy system 100 may provide therapy to manage symptoms of other patient conditions, such as, but not limited to, seizure disorders (e.g., epilepsy) or mood (or psychological) disorders (e.g., major depressive disorder (MDD), bipolar disorder, anxiety disorders, post-traumatic stress disorder, dysthymic disorder, and obsessive-compulsive disorder (OCD)). At least some of these disorders may be manifested in one or more patient movement behaviors. As described herein, a movement disorder or other neurodegenerative impairment may include symptoms such as, for example, muscle control impairment, motion impairment or other movement problems, such as rigidity, spasticity, bradykinesia, rhythmic hyperkinesia, nonrhythmic hyperkinesia, and akinesia. In some cases, the movement disorder may be a symptom of Parkinson's disease. However, the movement disorder may be attributable to other patient conditions. In some examples, a patient may be subject to two or more conditions that may be treated using different therapy modes and/or monitored using one or more different sensing modalities. For example, IMD 106 may switch between different sensing modality configurations for respective therapies delivered to treat the respective conditions.

Example therapy system 100 includes medical device programmer 104, implantable medical device (IMD) 106, lead extension 110, and leads 114A and 114B with respective sets of electrodes 116, 118. In the example shown in FIG. 1, electrodes 116, 118 of leads 114A, 114B are positioned to deliver electrical stimulation to a tissue site within brain 120, such as a deep brain site under the dura mater of brain 120 of patient 112. In some examples, delivery of stimulation to one or more regions of brain 120, such as the subthalamic nucleus, globus pallidus or thalamus, may be an effective treatment to manage movement disorders, such as Parkinson's disease. Some or all of electrodes 116, 118 also may be positioned to sense neurological brain signals within brain 120 of patient 112. In some examples, some of electrodes 116, 118 may be configured to sense neurological brain signals via sensing circuitry and the same or others of electrodes 116, 118 may be configured to deliver adaptive electrical stimulation to brain 120 via stimulation circuitry. In other examples, all of electrodes 116, 118 are configured to both sense neurological brain signals via the sensing circuitry and deliver adaptive electrical stimulation to brain 120 via the stimulation circuitry.

IMD 106 includes a therapy module (e.g., which may include processing circuitry, signal generation circuitry, stimulation circuitry, or other electrical circuitry configured to perform the functions attributed to IMD 106) that includes a stimulation generator configured to generate and deliver electrical stimulation therapy to patient 112 via a subset of electrodes 116, 118 of leads 114A and 114B, respectively. The subset of electrodes 116, 118 that are used to deliver electrical stimulation to patient 112, and, in some cases, the polarity of the subset of electrodes 116, 118, may be referred to as a stimulation electrode combination. As described in further detail below, the stimulation electrode combination can be selected for a particular patient 112 and target tissue site (e.g., selected based on the patient condition). The group of electrodes 116, 118 includes at least one electrode and can include a plurality of electrodes. In some examples, the plurality of electrodes 116 and/or 118 may have a complex electrode geometry such that two or more electrodes are located at different positions around the perimeter of the respective lead.

In some examples, the neurological signals sensed within brain 120 may reflect changes in electrical current produced by the sum of electrical potential differences across brain tissue. Examples of neurological brain signals include, but are not limited to, electrical signals generated from local field potentials (LFP) sensed within one or more regions of brain 120, such as an electroencephalogram (EEG) signal, or an electrocorticogram (EcoG) signal. Local field potentials, however, may include a broader genus of electrical signals within brain 120 of patient 112. In some examples, IMD 106 may sense evoked signals, such as ECAP signals or evoked resonant neural activity (ERNA) signals, that may be neural signals directly elicited by a delivered electrical stimulus. Each of these signals are different sensing modalities.

In some examples, the neurological brain signals that are used to select a stimulation electrode combination may be sensed within the same region of brain 120 as the target tissue site for the electrical stimulation. As previously indicated, these tissue sites may include tissue sites within anatomical structures such as the thalamus, subthalamic nucleus or globus pallidus of brain 120, as well as other target tissue sites. The specific target tissue sites and/or regions within brain 120 may be selected based on the patient condition. Thus, in some examples, the electrodes used for delivering electrical stimulation may be different than the electrodes used for sensing neurological brain signals. In other examples, the same electrodes may be used to deliver electrical stimulation and sense brain signals. However, this configuration would require the system to switch between stimulation generation and sensing circuitry and may reduce the time the system can sense brain signals.

Electrical stimulation generated by IMD 106 may be configured to manage a variety of disorders and conditions. In some examples, the stimulation generator of IMD 106 is configured to generate and deliver electrical stimulation pulses to patient 112 via electrodes of a selected stimulation electrode combination. However, in other examples, the stimulation generator of IMD 106 may be configured to generate and deliver a continuous wave signal, e.g., a sine wave or triangle wave. In either case, a stimulation generator within IMD 106 may generate the electrical stimulation therapy for DBS according to a therapy program that is selected at that given time in therapy. In examples in which IMD 106 delivers electrical stimulation in the form of stimulation pulses, a therapy program may include a set of therapy parameter values (e.g., stimulation parameters), such as a stimulation electrode combination for delivering stimulation to patient 112, pulse frequency, pulse width, and a current or voltage amplitude of the pulses. As previously indicated, the electrode combination may indicate the specific electrodes 116, 118 that are selected to deliver stimulation signals to tissue of patient 112 and the respective polarities of the selected electrodes.

IMD 106 may be implanted within a subcutaneous pocket above the clavicle, or, alternatively, on or within cranium 122 or at any other suitable site within patient 112. Generally, IMD 106 is constructed of a biocompatible material that resists corrosion and degradation from bodily fluids. IMD 106 may comprise a hermetic housing to substantially enclose components, such as a processor, therapy module, and memory.

As shown in FIG. 1, implanted lead extension 110 is coupled to IMD 106 via connector 108 (also referred to as a connector block or a header of IMD 106). In the example of FIG. 1, lead extension 110 traverses from the implant site of IMD 106 and along the neck of patient 112 to cranium 122 of patient 112 to access brain 120. In the example shown in FIG. 1, leads 114A and 114B (collectively “leads 114”) are implanted within the right and left hemispheres, respectively, of patient 112 in order deliver electrical stimulation to one or more regions of brain 120, which may be selected based on the patient condition or disorder controlled by therapy system 100. The specific target tissue site and the stimulation electrodes used to deliver stimulation to the target tissue site, however, may be selected, e.g., according to the identified patient behaviors and/or other sensed patient parameters. Other lead 114 and IMD 106 implant sites are contemplated. For example, IMD 106 may be implanted on or within cranium 122, in some examples. Or leads 114 may be implanted within the same hemisphere or IMD 106 may be coupled to a single lead implanted in a single hemisphere.

Existing lead sets include axial leads carrying ring electrodes disposed at different axial positions and so-called “paddle” leads carrying planar arrays of electrodes. Selection of electrode combinations within an axial lead, a paddle lead, or among two or more different leads presents a challenge to the clinician. In some examples, more complex lead array geometries may be used.

Although leads 114 are shown in FIG. 1 as being coupled to a common lead extension 110, in other examples, leads 114 may be coupled to IMD 106 via separate lead extensions or directly to connector 108. Leads 114 may be positioned to deliver electrical stimulation to one or more target tissue sites within brain 120 to manage patient symptoms associated with a movement disorder of patient 112. Leads 114 may be implanted to position electrodes 116, 118 at desired locations of brain 120 through respective holes in cranium 122. Leads 114 may be placed at any location within brain 120 such that electrodes 116, 118 are capable of providing electrical stimulation to target tissue sites within brain 120 during treatment. For example, electrodes 116, 118 may be surgically implanted under the dura mater of brain 120 or within the cerebral cortex of brain 120 via a burr hole in cranium 122 of patient 112, and electrically coupled to IMD 106 via one or more leads 114. Although leads 114 only show electrodes located at the distal end of the respective lead, electrodes may be placed at any location along the respective lead for stimulation and/or sensing purposes. For example different sensing modalities may utilize different sensing vectors that may utilize electrodes located at different anatomical locations. The lead may carry any of these electrodes or other sensors (e.g., accelerometers, biochemical sensors, etc.) along the housing of the lead and/or on any leads or leadlets that extend from the respective lead.

In the example shown in FIG. 1, electrodes 116, 118 of leads 114 are shown as ring electrodes. Ring electrodes may be used in DBS applications because they are relatively simple to program and are capable of delivering an electrical field to any tissue adjacent to electrodes 116, 118. In other examples, electrodes 116, 118 may have different configurations. For example, in some examples, at least some of the electrodes 116, 118 of leads 114 may have a complex electrode array geometry that is capable of producing shaped electrical fields. The complex electrode array geometry may include multiple electrodes (e.g., partial ring or segmented electrodes) around the outer perimeter of each lead 114, rather than one ring electrode. In this manner, electrical stimulation may be directed in a specific direction from leads 114 to enhance therapy efficacy and reduce possible adverse side effects from stimulating a large volume of tissue. In some examples, a housing of IMD 106 may include one or more stimulation and/or sensing electrodes. In alternative examples, leads 114 may have shapes other than elongated cylinders as shown in FIG. 1. For example, leads 114 may be paddle leads, spherical leads, bendable leads, or any other type of shape effective in treating patient 112 and/or minimizing invasiveness of leads 114.

In the example shown in FIG. 1, IMD 106 includes a memory to store a plurality of therapy programs that each define a set of therapy parameter values. In some examples, IMD 106 may select a therapy program from the memory based on various parameters, such as sensed patient parameters and the identified patient behaviors. IMD 106 may generate electrical stimulation based on the selected therapy program to manage the patient symptoms associated with a movement disorder. IMD 106 may also store the different sensing modality configurations in the memory or a different memory.

External programmer 104 wirelessly communicates with IMD 106 as needed to provide or retrieve therapy information. Programmer 104 is an external computing device that the user, e.g., a clinician and/or patient 112, may use to communicate with IMD 106. For example, programmer 104 may be a clinician programmer that the clinician uses to communicate with IMD 106 and program one or more therapy programs for IMD 106. Alternatively, programmer 104 may be a patient programmer that allows patient 112 to select programs, view and modify therapy parameters, and/or view and/or select sensing modalities for any sensing modality configuration. The clinician programmer may include more programming features than the patient programmer. In other words, more complex or sensitive tasks may only be allowed by the clinician programmer to prevent an untrained patient from making undesirable changes to IMD 106.

When programmer 104 is configured for use by the clinician, programmer 104 may be used to transmit initial programming information to IMD 106. This initial information may include hardware information, such as the type of leads 114 and the electrode arrangement, the position of leads 114 within brain 120, the configuration of electrode array 116, 118, initial programs defining therapy parameter values, sensing modality configurations, and any other information the clinician desires to program into IMD 106. Programmer 104 may also be capable of completing functional tests (e.g., measuring the impedance of electrodes 116, 118 of leads 114). In addition, or as an alternative, to programmer 104, a different external computing device may perform any of the functionality of programmer 104. The external computing device may be a networked device and in communication with IMD 106 directly or via programmer 104.

The clinician may also store therapy programs within IMD 106 with the aid of programmer 104. During a programming session, the clinician may determine one or more therapy programs that may provide efficacious therapy to patient 112 to address symptoms associated with the patient condition, and, in some cases, specific to one or more different patient states, such as a sleep state, movement state or rest state. For example, the clinician may select one or more stimulation electrode combination with which stimulation is delivered to brain 120. During the programming session, the clinician may evaluate the efficacy of the specific program being evaluated based on feedback provided by patient 112 or based on one or more physiological parameters of patient 112 (e.g., muscle activity, muscle tone, rigidity, tremor, etc.) which may be input by a user and/or sensed by any sensing modality provided by IMD 106 or another associated device that may be implanted in, worn on, or otherwise monitoring patient 112. Alternatively, identified patient behavior from video information may be used as feedback during the initial and subsequent programming sessions. Programmer 104 may assist the clinician in the creation/identification of therapy programs by providing a methodical system for identifying potentially beneficial therapy parameter values.

Programmer 104 may also be configured for use by patient 112. When configured as a patient programmer, programmer 104 may have limited functionality (compared to a clinician programmer) in order to prevent patient 112 from altering critical functions of IMD 106 or applications that may be detrimental to patient 112. In this manner, programmer 104 may only allow patient 112 to adjust values for certain therapy parameters or set an available range of values for a particular therapy parameter.

Programmer 104 may also provide an indication to patient 112 when therapy is being delivered, when patient input has triggered a change in therapy or when the power source within programmer 104 or IMD 106 needs to be replaced or recharged. For example, programmer 112 may include an alert LED, may flash a message to patient 112 via a programmer display, generate an audible sound or somatosensory cue to confirm patient input was received, e.g., to indicate a patient state or to manually modify a therapy parameter.

Therapy system 100 may be implemented to provide chronic stimulation therapy to patient 112 over the course of several months or years. However, system 100 may also be employed on a trial basis to evaluate therapy before committing to full implantation. If implemented temporarily, some components of system 100 may not be implanted within patient 112. For example, patient 112 may be fitted with an external medical device, such as a trial stimulator, rather than IMD 106. The external medical device may be coupled to percutaneous leads or to implanted leads via a percutaneous extension. If the trial stimulator indicates DBS system 100 provides effective treatment to patient 112, the clinician may implant a chronic stimulator within patient 112 for relatively long-term treatment.

Although IMD 104 is described as delivering electrical stimulation therapy to brain 120, IMD 106 may be configured to direct electrical stimulation to other anatomical regions of patient 112 in other examples. In other examples, system 100 may include an implantable drug pump in addition to, or in place of, IMD 106. Further, an IMD may provide other electrical stimulation such as spinal cord stimulation to treat a movement disorder.

According to the techniques of the disclosure, system 100 can be configured to define a homeostatic window and a therapeutic window for delivering adaptive DBS to patient 112. System 100 may adaptively deliver electrical stimulation and adjust one or more parameters defining the electrical stimulation within a parameter range defined by upper and lower limits of the therapeutic window based on the activity of the sensed signal (e.g., LFP signal) within the homeostatic window or any other single or combination of sensing modalities. For example, system 100 may adjust the one or more parameters defining the electrical stimulation in response to the sensed signal falling below the lower threshold or exceeding the upper threshold of the homeostatic window, but may not adjust the one or more parameters defining the electrical stimulation such that they fall below the lower limit or exceed the upper limit of the therapeutic window.

In one example, external programmer 104 issues commands to IMD 106 causing IMD 106 to deliver electrical stimulation therapy via electrodes 116, 118 via leads 114. As described above, the therapeutic window defines an upper bound and a lower bound for one or more parameters defining the delivery of electrical stimulation therapy to patient 112. For example, the one or more parameters include a current amplitude (for a current-controlled system) or a voltage amplitude (for a voltage-controlled system), a pulse rate or frequency, and a pulse width. In examples where the electrical stimulation is delivered according to a “burst” of pulses, or a series of electrical pulses defined by an “on-time” and an “off-time,” the one or more parameters may further define one or more of a number of pulses per burst, an on-time, and an off-time. In one example, the therapeutic window defines an upper bound and a lower bound for one or more parameters, such as upper and lower threshold for a current amplitude of the electrical stimulation therapy (in current-controlled systems) or upper and lower threshold of a voltage amplitude of the electrical stimulation therapy (in voltage-controlled systems). While the examples herein are typically given with respect to adjusting a voltage amplitude or a current amplitude, the techniques herein may equally be applied to a homeostatic window and a therapeutic window using other parameters, such as, e.g., pulse rate or pulse width. Example implementations of the therapeutic window are provided in further detail below.

Typically, a patient programmer 104 may not have access to adjustments to any thresholds or limits for sensing or stimulation related to adaptive DBS. For example, patient programmer 104 may only enable a patient to adjust a stimulation parameter value between limits set by the clinician programmer. However, in other examples, system 100 may provide adaptive DBS by permitting a patient 112, e.g., via a patient programmer 104, to indirectly adjust the activation, deactivation, and magnitude of the electrical stimulation by adjusting the lower and upper threshold of the homeostatic window. In one example, the patient programmer 104 may only be enabled to adjust an upper or lower threshold a small magnitude or percentage of the clinician-set value. In another example, by adjusting one or both thresholds of the homeostatic window, patient 112 may adjust the point at which the sensed signal deviates from the homeostatic window, triggering system 100 to adjust one or more parameters of the electrical stimulation within a parameter range defined by the lower and upper threshold of the therapeutic window.

In some examples, a patient may provide feedback, e.g., via programmer 104, to adjust one or both threshold of the homeostatic window. In another example, programmer 104 and/or IMD 106 may automatically adjust one or both threshold of the homeostatic window, as well as one or more parameters of the electrical stimulation within the parameter range defined by the lower and upper threshold of the therapeutic window. For example, IMD 106 may adjust the delivery of adaptive DBS by automatically adjusting one or more thresholds (e.g., an upper and a lower threshold in some examples) of the homeostatic window, e.g., in response to a physiological parameter sensed by one or more sensors 109 of system 100. As a further example, programmer 104 and/or IMD 106 may automatically adjust one or more thresholds of the homeostatic window based on one or more physiological or neurological signals of patient 112 sensed by IMD 106. For example, in response to deviations in the signal of the patient outside of the homeostatic window, system 100 (e.g., IMD 106 or programmer 104) may automatically adjust one or more parameters defining the electrical stimulation therapy delivered to the patient in a manner that is proportional to the magnitude of the sensed signal and within the therapeutic window defining lower and upper thresholds for the one or more parameters. The adjustment to the one or more stimulation therapy parameters based on the deviation of the sensed signal may be proportional or inversely proportional to the magnitude of the signal.

Hence, in some examples, system 100, via programmer 104 or IMD 106, may adjust one or more parameters of the electrical stimulation, such as voltage or current amplitude, within the therapeutic window based on patient input that adjusts the homeostatic window, or based on one or more signals, such as sensed physiological parameters or sensed neurological signals, or a combination of two or more of the above. In particular, system 100 may adjust a parameter of the electrical stimulation, automatically and/or in response to patient input that adjusts the homeostatic window, provided the value of the electrical stimulation parameter is constrained to remain within a range specified by the upper and lower threshold of the therapeutic window. This range may be considered to include the upper and lower threshold themselves.

In some examples where system 100 adjusts multiple parameters of the electrical stimulation, system 100 may adjust at least one of a voltage amplitude or current amplitude, a stimulation frequency, a pulse width, or a selection of electrodes, and the like. In such an example, the clinician may set an order or sequence for adjustment of the parameters (e.g., adjust voltage amplitude or current amplitude, then adjust stimulation frequency, and then adjust the selection of electrodes). In other examples, system 100 may randomly select a sequence of adjustments to the multiple parameters. In either example, system 100 may adjust a value of a first parameter of the parameters of the electrical stimulation. If the signal does not exhibit a response to the adjustment of the first parameter, system 100 may adjust a value of a second parameter of the parameters of the electrical stimulation, and so on until the signal returns to within the homeostatic window.

IMD 106 may adjust one or more parameters that at least partially define DBS therapy based on one or more sensing modalities specified in the sensing modality configuration for that DBS therapy mode. For example, to adaptively adjust DBS based on a neurological signal (e.g., a sensing modality), for example, two or more electrodes 116, 118 of IMD 106 may be configured to monitor a neurological signal (e.g., an LFP signal) of patient 112. In some examples, at least one of electrodes 116, 118 may be provided on a housing of IMD 106, providing a unipolar stimulation and/or sensing configuration. In one example, the neurological signal is a signal within a Beta frequency band of brain 120 of patient 112. For example, neurological signals within the Beta frequency band of patient 112 may correlate to one or more symptoms of Parkinson's disease in patient 112. Generally speaking, neurological signals within the Beta frequency of patient 112 may be approximately proportional to the severity of the symptoms of patient 112. For example, as tremor induced by Parkinson's disease increases, neurological signals within the Beta frequency of patient 112 increase (e.g., magnitude of the signal and/or spectral power). Moreover, neurological signals within the Beta frequency are considered proportional because system 100 may be configured such that an increase in signal magnitude may trigger system 100 to increase delivered stimulation therapy magnitude according to disclosed techniques. Similarly, as tremor induced by Parkinson's disease decreases, neurological signals within the Beta frequency of patient 112 decrease (e.g., magnitude of the signal and/or spectral power), and the decrease may trigger system 100 to decrease the magnitude of delivered stimulation.

In some examples, each of a sensor within IMD 106 is an accelerometer, a bonded piezoelectric crystal, a mercury switch, or a gyro. In some examples, these sensors may be or be part of a different sensing modality that provides a signal that indicates a respective physiological parameter of the patient, which in turn varies as a function of patient activity. For example, the device may monitor a signal that indicates the heart rate, electrocardiogram (ECG) morphology, electroencephalogram (EEG) morphology, respiration rate, respiratory volume, core temperature, subcutaneous temperature, or muscular activity of the patient. In some examples, sensing circuitry that senses LFP signals may be used to sense other electrical activity that can be processed for a different sensing modality, such as an EEG or ECG signal.

In some examples, the sensors of one or more sensing modalities can be configured to generate a signal both as a function of patient activity and patient posture. For example, accelerometers, gyros, or magnetometers may generate respective signals that indicate both the activity and the posture of a patient 112. External programmer 104 may use such information regarding posture to determine whether external programmer 104 should perform adjustments to the therapeutic window, when to cycle stimulation on and off, when to switch to different sensing modality configurations, or any other purpose.

For example, in order to identify posture, the sensors such as accelerometers may be oriented substantially orthogonally with respect to each other. In addition to being oriented orthogonally with respect to each other, each of the sensors used to detect the posture of a patient 112 may be substantially aligned with an axis of the body of a patient 112. When accelerometers, for example, are aligned in this manner, the magnitude and polarity of DC components of the signals generate by the accelerometers indicate the orientation of the patient relative to the Earth's gravity, e.g., the posture of a patient 112. Further information regarding use of orthogonally aligned accelerometers to determine patient posture may be found in a commonly assigned U.S. Pat. No. 5,593,431, which issued to Todd J. Sheldon, the entire content of which is incorporated by reference herein.

Other sensors that may generate a signal (e.g., a sensing modality) that indicates the posture of a patient 112 include electrodes that generate a signal as a function of electrical activity within muscles of a patient 112, e.g., an electromyogram (EMG) signal, or a bonded piezoelectric crystal that generates a signal as a function of contraction of muscles. Electrodes or bonded piezoelectric crystals may be implanted in the legs, buttocks, chest, abdomen, or back of a patient 112, and coupled to one or more of external programmer 104 and IMD 106 wirelessly or via one or more leads. Alternatively, electrodes may be integrated in a housing of the IMD 106 or piezoelectric crystals may be bonded to the housing when IMD 106 is implanted in the buttocks, chest, abdomen, or back of a patient 112. The signals generated by such sensors when implanted in these locations may vary based on the posture of a patient 112, e.g., may vary based on whether the patient is standing, sitting, or lying down.

Further, the posture of a patient 112 may affect the thoracic impedance of the patient. Consequently, sensors may include an electrode pair, including one electrode integrated with the housing of IMDs 106 and one of electrodes 116, 118, that generate a signal as a function of the thoracic impedance of a patient 112, and IMD 106 may detect the posture or posture changes of a patient 112 based on the signal. In one example (not depicted), the electrodes of the pair may be located on opposite sides of the patient's thorax. For example, the electrode pair may include electrodes located proximate to the spine of a patient for delivery of SCS therapy, and IMD 106 with an electrode integrated in its housing may be implanted in the abdomen or chest of patient 112. As another example, IMD 106 may include electrodes implanted to detect thoracic impedance in addition to leads 114 implanted within the brain of patient 112. The posture or posture changes may affect the delivery of DBS or SCS therapy to patient 112 for the treatment of any type of neurological disorder, and may also be used to detect patient sleep, as described herein.

Additionally, changes of the posture of a patient 112 may cause pressure changes with the cerebrospinal fluid (CSF) of the patient. Consequently, sensors may include pressure sensors coupled to one or more intrathecal or intracerebroventricular catheters, or pressure sensors coupled to IMDs 106 wirelessly or via one of leads 114. CSF pressure changes associated with posture changes may be particularly evident within the brain of the patient, e.g., may be particularly apparent in an intracranial pressure (ICP) waveform.

Accordingly, in some examples, instead of, or in addition to, monitoring a neurological signal of the patient, the system 100 monitors one or more signals from sensors indicative of a magnitude of a physiological parameter of patient 112. Any of the sensing modalities may be used individually or together in respective sensing modality configurations, and system 100 may switch between different sensing modality configurations as needed based on changes to a therapy mode, changes to patient condition, detected error with a sensing modality, to confirm a sense event of the patient, or any other reasons. Upon detecting that one or more signals from sensors exceed the upper bound of the homeostatic window, the system 100 can be configured to increase stimulation at a maximum ramp rate determined by the clinician until one or more signals from sensors return to within the homeostatic window, or until the magnitude of the electrical stimulation reaches an upper limit of a therapeutic window determined by the clinician. Similarly, upon detecting that one or more signals from sensors falls below the lower bound of the homeostatic window, the system decreases stimulation at a maximum ramp rate determined by the clinician until one or more signals from sensors return to within the homeostatic window, or until the magnitude of the electrical stimulation reaches a lower limit of a therapeutic window determined by the clinician. Upon detecting that one or more signals from sensors are within the threshold of the homeostatic window, the system holds the magnitude of the electrical stimulation constant.

Such a system 100 for delivering adaptive DBS to the patient according to different sensing modality configurations may provide advantages over other techniques that use a neurological signal as a threshold in that the techniques of the disclosure allow an IMD to control delivery of therapy using hysteresis. In other words, such a system 100 uses the physiological parameter of the patient from respective sensing modalities to create a control loop for not only controlling the delivery of therapy, but also controlling the magnitude of the delivered therapy. Such a system may be less intrusive on the activity of a patient because the system 100 adapts the stimulation to the current needs of the patient, needs of therapy, and or the needs of the system, and thus may reduce the side effects that the patient experiences.

Further, such a system 100 may use, as part of any sensing modality or sensing modality configurations, external sensors, such as accelerometers, instead of internal sensors, such as electrodes, to detect symptoms of the disease of the patient and control adjustments to the magnitude of one or more parameters of the therapy. For example, the system 100 may use a wrist sensor to detect wrist flexion or tremor of a patient suffering from Parkinson's disease. Thus, such an IMD the monitoring of a physiological parameter may be less invasive than other IMD systems because the system of the present disclosure may not require sensing electrodes to be implanted in the brain of the patient 112.

In some circumstances, system 100, as described herein, may deliver, based on the upper and lower threshold of the homeostatic window, a lower magnitude of electrical stimulation than patient 112 requires to prevent breakthrough of his or her symptoms. For example, a patient receiving therapy from an IMD 106 that controls delivery of electrical stimulation therapy using the homeostatic window may, in certain circumstances, experience results that are less optimal than if the patient received continuous electrical stimulation therapy at a maximum therapy magnitude. To prevent this occurrence, system 100 may determine a value for the at least one electrical stimulation parameter as defined by the homeostatic window, as described above. Further, the IMD 106 of system 100 may increase the value for the at least one electrical stimulation parameter by a bias amount greater than the determined magnitude defined by the homeostatic window so as to further prevent breakthrough of the symptoms of patient 112. Thus, system 100 may avoid delivering electrical stimulation therapy that is of a magnitude that may be insufficient for prevention of symptom breakthrough.

The architecture of system 100 illustrated in FIG. 1 is shown as an example. The techniques as set forth in this disclosure may be implemented in the example system 100 of FIG. 1, as well as other types of systems not described specifically herein. For example, a clinician may determine the upper threshold and lower threshold of the homeostatic window. In other examples, one of the external programmer 104 and IMD 104 determines the upper threshold and lower threshold of the homeostatic window. Furthermore, either external programmer 104 or IMD 106 may receive the signal representative of the signal of patient 112 and determine an adjustment to one or more parameters defining the electrical stimulation therapy that IMD 106 delivers to patient 112. Nothing in this disclosure should be construed so as to limit the techniques of this disclosure to the example architecture illustrated by FIG. 1.

In some examples, system 100 (which may include IMD 106 and/or additional devices such as programmer 104) may include a memory configured to store a plurality of different sensing modality configurations. Each sensing modality configuration of the plurality of sensing modality configurations defines at least one sensing modality of a plurality of sensing modalities. System 100 also includes processing circuitry configured to control stimulation generation circuitry to deliver electrical stimulation therapy based on a first sensing modality configuration of the plurality of different sensing modality configurations. The processing circuitry can also determine a change to at least one of a patient state or a therapy mode and responsive to determining the change, select a second sensing modality configuration of the plurality of different sensing modality configurations. Then, the processing circuitry can control the stimulation generation circuitry to deliver the electrical stimulation therapy based on the second sensing modality configuration instead of the first sensing modality configuration.

The plurality of sensing modalities comprise at least one of a biochemical sensing modality, an electrophysical sensing modality, a patient motion sensing modality, or a posture state sensing modality. The biochemical sensing modality may be configured to sense a presence of one or more neurotransmitters, one or more medications, or one or more physiological molecules generated in response to the medications, in a patient. A sensor may be constructed of a polymer or other material that may or may not coated with a material that interacts with molecules in the patient, and electrical characteristics of the material. For example, the biochemical sensing modality may include one or more biochemical sensors configured to be implanted within the patient, either carried by one of leads 114 or a different tethered or wireless sensor in communication with IMD 106.

The electrophysical sensing modality may be configured to sense any electrical signal generated by tissue. Example electrophysical sensing modalities may include at least one of a local field potential sensing modality, an evoked neural response sensing modality, a muscular sensing modality (e.g., electromyogram (EMG)), electroencephalogram (EEG), electrocardiogram (ECG), etc.), or a voltammetry sensing modality. LFP and evoked signals may be electrical potentials within tissue that are detected between electrodes via sensing circuitry. Voltammetry may include driving a voltage to tissue between two electrodes while the system monitors the current required to deliver provide that voltage. Various chemicals in the tissue subject to the voltage may change how the current needs to be applied to the electrodes. Neurotransmitters such as dopamine, serotonin, and norepinephrine, may be detected (e.g., presence or quantity) using voltammetry sensing techniques. The patient motion sensing modality may include the detection of patient movement using sensors such as one or more accelerometers and/or EMG signals indicative of muscle activity. The posture state sensing modality may include detection of a position of the patient using sensors such as accelerometers, pressure sensors, or any other sensors. Described in another way, an accelerometer sensing modality may be configured to sense at least one of an activity or a posture of the patient.

In some examples, a sensing modality configuration may only specify a single sensing modality to be used as feedback for controlling stimulation therapy. In other examples, a single sensing modality configuration may include two, three, four, or more different sensing modalities. These different sensing modalities may all be used as feedback to control the same stimulation therapy mode. In some examples, each sensing modality is used as a different factor for feedback on the therapy, such as one modality may be used to cycle stimulation on and off while another modality may be used to increase and decrease stimulation intensity (e.g., amplitude and/or pulse width of electrical stimulation pulses). In other examples, at least one sensing modality within the sensing modality configuration may be used to confirm output from another sensing modality. For example, the system may not adjust stimulation from a first sensing modality until the system can confirm that the output from the first sensing modality (e.g., LFP signal) is consistent with or agrees with the output from a second sensing modality (e.g., an accelerometer signal).

System 100 may switch between different sensing modality configurations in response to detected changes to various aspects associated with therapy, such as a different therapy mode or different patient state. A therapy mode may refer to a type of stimulation delivered via a certain electrode combination, specific interleaved stimulation pulse trains, different pulse frequencies, medication delivery, etc. A patient state may refer to a disease state, such as a change in the progression (e.g., severity or type of symptoms) of the disease. A patient state may also refer to a physiological condition separate from the disease (e.g., food intake, stress level, etc.), activity level of the patient, and/or posture state (e.g., how the patient is oriented with respect to gravity). The patient state may also include the sleep state of the patient, which may inform what type of therapy to deliver or whether to turn stimulation on or off. In this manner, different sensing modality configurations may be assigned to different patient states of the patient to enable the system to switch between sensing modality configurations in response to detecting which patient state the patient currently is in.

In one example, system 100 may switch between sensing modality configurations based on the pulse frequency, for example, of the stimulation delivered according to the therapy mode currently used by the system. The system may be configured to control sensing circuitry to sense LFPs as one sensing modality of the plurality of sensing modalities during one or more first therapy modes that include electrical stimulation therapy using first stimulation pulse frequencies greater than a threshold frequency for sensing ECAP signals. The threshold frequency may indicate the threshold above which the system cannot sense ECAP signals without interference by the next pulse being delivered. Therefore, the system may use LFPs to control stimulation in this therapy mode. In addition, the processing circuitry can control the sensing circuitry to sense ECAP signals as another sensing modality of the plurality of sensing modalities during one or more second therapy modes that include electrical stimulation therapy using second stimulation pulse frequencies less than the threshold frequency for sensing the ECAP signals.

System 100 may also switch to different sensing modality configurations, or replace a sensing modality within a sensing modality configuration, in response to detecting an error with a sensing modality. The error may be determined in a current sensing modality by determining the output from the sensing modality signal being outside of an expected range, lack of change over a period of time, excessive frequency of amplitude change, or any other indication of an error in sensing or artifact in the sensed signal. IMD 106 can then continue controlling stimulation using the switched sensing modality.

System 100 may also confirm a signal from one sensing modality with a different sensing modality to ensure that the data used for controlling stimulation is accurate. For example, IMD 106 may detect a first sense event using a first sensing modality. Then, IMD 106 can confirm presence of the first sense event based on a second sensing modality different than the first sensing modality. The first sense event may be an LFP signal event that can be corroborated, or confirmed, by a movement of the patient that is detected by an accelerometer, for example. Then, IMD 106 can, responsive to confirming the presence of the first sense event, adjust a parameter that at least partially defines the electrical stimulation therapy. This process may be performed for one, some, or all of the sensing modalities used to control stimulation. In some examples, the system may trigger the confirmation process in response to detecting that the first sense event or some other aspect of the first sense signal may be outside of an expected range or unexpected based on other patient conditions or therapy delivery.

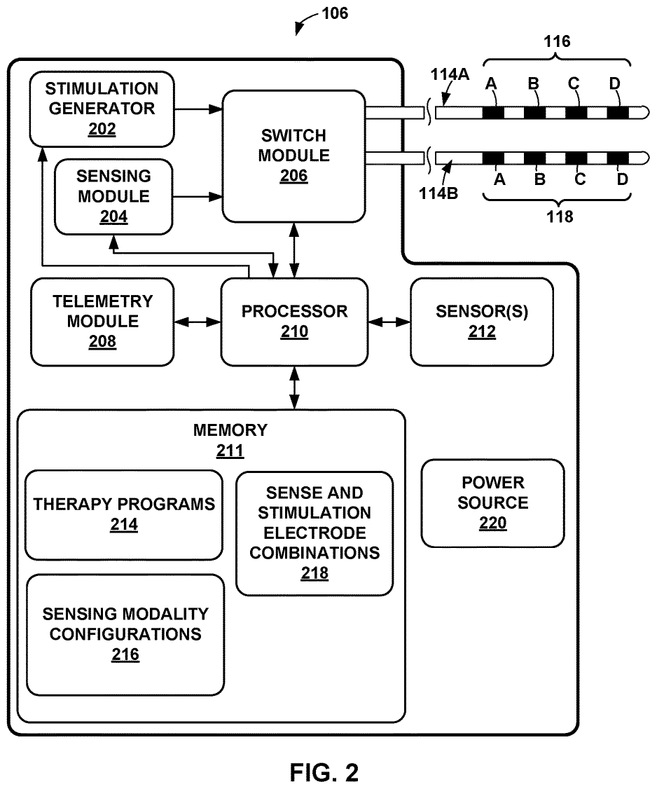

FIG. 2 is a block diagram of the example IMD 106 of FIG. 1 for delivering adaptive deep brain stimulation therapy. In the example shown in FIG. 2, IMD 106 includes processor 210, memory 211, stimulation generator 202, sensing module 204, switch module 206, telemetry module 208, sensor 212, and power source 220. Each of these modules may be or include electrical circuitry configured to perform the functions attributed to each respective module. For example, processor 210 may include processing circuitry, switch module 206 may include switch circuitry, sensing module 204 may include sensing circuitry, and telemetry module 208 may include telemetry circuitry. Switch module 204 may not be necessary for multiple current source and sink configurations. Memory 211 may include any volatile or non-volatile media, such as a random-access memory (RAM), read only memory (ROM), non-volatile RAM (NVRAM), electrically erasable programmable ROM (EEPROM), flash memory, and the like. Memory 211 may store computer-readable instructions that, when executed by processor 210, cause IMD 106 to perform various functions. Memory 211 may be a storage device or other non-transitory medium.

In the example shown in FIG. 2, memory 211 stores therapy programs 214 and sense electrode combinations and associated stimulation electrode combinations 218 in separate memories within memory 211 or separate areas within memory 211. Each stored therapy program 214 defines a particular set of electrical stimulation parameters (e.g., a therapy parameter set), such as a stimulation electrode combination, electrode polarity, current or voltage amplitude, pulse width, and pulse rate. In some examples, individual therapy programs may be stored as a therapy group, which defines a set of therapy programs with which stimulation may be generated. The stimulation signals defined by the therapy programs of the therapy group may be delivered together on an overlapping or non-overlapping (e.g., time-interleaved) basis. Memory 211 may also store one or more sensing modality configurations that will be used for respective operations situations, such as respective therapy modes or patient states. The sensing modality configurations 216 may indicate which one or more sensing modalities are to be used within each sensing modality configuration, how each sensing modality is used as feedback for each sensing modality configuration, which therapy mode or patient state is associated with which sensing modality configuration, or any other associated information.

Sense and stimulation electrode combinations 218 stores sense electrode combinations and associated stimulation electrode combinations. As described above, in some examples, the sense and stimulation electrode combinations may include the same subset of electrodes 116, 118, a housing of IMD 106 functioning as an electrode, or may include different subsets or combinations of such electrodes. Thus, memory 211 can store a plurality of sense electrode combinations and, for each sense electrode combination, store information identifying the stimulation electrode combination that is associated with the respective sense electrode combination. The associations between sense and stimulation electrode combinations can be determined, e.g., by a clinician or automatically by processor 210. In some examples, corresponding sense and stimulation electrode combinations may comprise some or all of the same electrodes. In other examples, however, some or all of the electrodes in corresponding sense and stimulation electrode combinations may be different. For example, a stimulation electrode combination may include more electrodes than the corresponding sense electrode combination in order to increase the efficacy of the stimulation therapy. In some examples, as discussed above, stimulation may be delivered via a stimulation electrode combination to a tissue site that is different than the tissue site closest to the corresponding sense electrode combination but is within the same region, e.g., the thalamus, of brain 120 in order to mitigate any irregular oscillations or other irregular brain activity within the tissue site associated with the sense electrode combination.

Stimulation generator 202, under the control of processor 210, generates stimulation signals for delivery to patient 112 via selected combinations of electrodes 116, 118. An example range of electrical stimulation parameters believed to be effective in DBS to manage a movement disorder of patient include:
1. Pulse Rate, i.e., Frequency: between approximately 40 Hertz and approximately 500 Hertz, such as between approximately 40 to 185 Hertz or such as approximately 140 Hertz.
2. In the case of a voltage controlled system, Voltage Amplitude: between approximately 0.1 volts and approximately 50 volts, such as between approximately 2 volts and approximately 3 volts.
3. In the alternative case of a current controlled system, Current Amplitude: between approximately 0.2 milliamps to approximately 100 milliamps, such as between approximately 1.3 milliamps and approximately 2.0 milliamps.
4. Pulse Width: between approximately 10 microseconds and approximately 5000 microseconds, such as between approximately 100 microseconds and approximately 1000 microseconds, or between approximately 180 microseconds and approximately 450 microseconds.

Accordingly, in some examples, stimulation generator 202 generates electrical stimulation signals in accordance with the electrical stimulation parameters noted above, subject to application of the upper and lower threshold of a therapeutic window to one or more of the parameters, such that an applicable parameter resides within the range prescribed by the window. Other ranges of therapy parameter values may also be useful, and may depend on the target stimulation site within patient 112. While stimulation pulses are described, stimulation signals may be of any form, such as continuous-time signals (e.g., sine waves) or the like.

Processor 210 may include fixed function processing circuitry and/or programmable processing circuitry, and may comprise, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processor 210 herein may be embodied as firmware, hardware, software or any combination thereof. Processor 210 may control stimulation generator 202 according to therapy programs 214 stored in memory 211 to apply particular stimulation parameter values specified by one or more of programs, such as voltage amplitude or current amplitude, pulse width, or pulse rate.

In the example shown in FIG. 2, the set of electrodes 116 includes electrodes 116A, 116B, 116C, and 116D, and the set of electrodes 118 includes electrodes 118A, 118B, 118C, and 118D. Processor 210 also controls switch module 206 to apply the stimulation signals generated by stimulation generator 202 to selected combinations of electrodes 116, 118. In particular, switch module 204 may couple stimulation signals to selected conductors within leads 114, which, in turn, deliver the stimulation signals across selected electrodes 116, 118. Switch module 206 may be a switch array, switch matrix, multiplexer, or any other type of switching module configured to selectively couple stimulation energy to selected electrodes 116, 118 and to selectively sense neurological brain signals with selected electrodes 116, 118. Hence, stimulation generator 202 is coupled to electrodes 116, 118 via switch module 206 and conductors within leads 114. In some examples, however, IMD 106 does not include switch module 206.

Stimulation generator 202 may be a single channel or multi-channel stimulation generator. In particular, stimulation generator 202 may be capable of delivering a single stimulation pulse, multiple stimulation pulses, or a continuous signal at a given time via a single electrode combination or multiple stimulation pulses at a given time via multiple electrode combinations. In some examples, however, stimulation generator 202 and switch module 206 may be configured to deliver multiple channels on a time-interleaved basis. For example, switch module 206 may serve to time divide the output of stimulation generator 202 across different electrode combinations at different times to deliver multiple programs or channels of stimulation energy to patient 112. Alternatively, stimulation generator 202 may comprise multiple voltage or current sources and sinks that are coupled to respective electrodes to drive the electrodes as cathodes or anodes. In this example, IMD 106 may not require the functionality of switch module 206 for time-interleaved multiplexing of stimulation via different electrodes.

Electrodes 116, 118 on respective leads 114 may be constructed of a variety of different designs. For example, one or both of leads 114 may include two or more electrodes at each longitudinal location along the length of the lead, such as multiple electrodes at different perimeter locations around the perimeter of the lead at each of the locations A, B, C, and D. On one example, the electrodes may be electrically coupled to switch module 206 via respective wires that are straight or coiled within the housing the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 114. These and other constructions may be used to create a lead with a complex electrode geometry.

Although sensing module 204 is incorporated into a common housing with stimulation generator 202 and processor 210 in FIG. 2, in other examples, sensing module 204 may be in a separate housing from IMD 106 and may communicate with processor 210 via wired or wireless communication techniques. Example neurological brain signals include, but are not limited to, a signal generated from local field potentials (LFPs) within one or more regions of brain 28. EEG and ECOG signals are other examples of electrical signals that may be measured within brain 120 or by electrodes placed in other locations with respect to brain 120.

Sensor 212 may include one or more sensing elements that sense values of a respective patient parameter. Any of these sensing elements or sensors may provide a sensing modality for IMD 106. For example, sensor 212 may include one or more accelerometers, optical sensors, chemical sensors, temperature sensors, pressure sensors, or any other types of sensors. Sensor 212 may output patient parameter values that may be used as feedback to control delivery of therapy, according to the selected sensing modality configuration. IMD 106 may include additional sensors within the housing of IMD 106 and/or coupled via one of leads 114 or other leads. In addition, IMD 106 may receive sensor signals wirelessly from remote sensors via telemetry module 208, for example. In some examples, one or more of these remote sensors may be external to patient (e.g., carried on the external surface of the skin, attached to clothing, or otherwise positioned external to the patient).

Telemetry module 208 supports wireless communication between IMD 106 and an external programmer 104 or another computing device under the control of processor 210. Processor 210 of IMD 106 may receive, as updates to programs, values for various stimulation parameters such as magnitude and electrode combination, from programmer 104 via telemetry module 208. The updates to the therapy programs may be stored within therapy programs 214 portion of memory 211. Telemetry module 208 in IMD 106, as well as telemetry modules in other devices and systems described herein, such as programmer 104, may accomplish communication by radiofrequency (RF) communication techniques. In addition, telemetry module 208 may communicate with external medical device programmer 104 via proximal inductive interaction of IMD 106 with programmer 104. Accordingly, telemetry module 208 may send information to external programmer 104 on a continuous basis, at periodic intervals, or upon request from IMD 106 or programmer 104.

Power source 220 delivers operating power to various components of IMD 106. Power source 220 may include a small rechargeable or non-rechargeable battery and a power generation circuit to produce the operating power. Recharging may be accomplished through proximal inductive interaction between an external charger and an inductive charging coil within IMD 220. In some examples, power requirements may be small enough to allow IMD 220 to utilize patient motion and implement a kinetic energy-scavenging device to trickle charge a rechargeable battery. In other examples, traditional batteries may be used for a limited period of time.

According to the techniques of the disclosure, processor 210 of IMD 106 delivers, electrodes 116, 118 interposed along leads 114 (and optionally switch module 206), electrical stimulation therapy to patient 112. The adaptive DBS therapy is defined by one or more therapy programs 214 having one or more parameters stored within memory 211. For example, the one or more parameters include a current amplitude (for a current-controlled system) or a voltage amplitude (for a voltage-controlled system), a pulse rate or frequency, and a pulse width, or quantity of pulses per cycle. In examples where the electrical stimulation is delivered according to a “burst” of pulses, or a series of electrical pulses defined by an “on-time” and an “off-time,” the one or more parameters may further define one or more of a number of pulses per burst, an on-time, and an off-time. In one example, the therapeutic window defines an upper limit and a lower limit for a voltage amplitude of the electrical stimulation therapy. In another example, the therapeutic window defines an upper limit and a lower limit for a current amplitude of the electrical stimulation therapy. In particular, a parameter of the electrical stimulation therapy, such as voltage or current amplitude, is constrained to a therapeutic window having an upper limit and a lower limit, such that the voltage or current amplitude may be adjusted provided the amplitude remains greater than or equal to the lower limit and less than or equal to the upper limit. It is noted that a single limit may be used in some examples.

In one example, processor 210, via electrodes 116, 118 of IMD 106, monitors the behavior of a signal of patient 112 that correlates to one or more symptoms of a disease of patient 112 within a homeostatic window. Processor 210, via electrodes 116, 118, delivers to patient 112 adaptive DBS and may adjust one or more parameters defining the electrical stimulation within a parameter range defined by lower and upper thresholds of a therapeutic window based on the activity of the sensed signal within the homeostatic window.

In one example, the signal is a neurological signal (e.g., a LFP signal) within the Beta frequency band of brain 120 of patient 112. The signal within the Beta frequency band of patient 112 may correlate to one or more symptoms of Parkinson's disease in patient 112. Generally speaking, neurological signals within the Beta frequency band of patient 112 may be approximately proportional to the severity of the symptoms of patient 112. For example, as tremor induced by Parkinson's disease increases, one or more of electrodes 116, 118 detect an increase in the magnitude of neurological signals within the Beta frequency band of patient 112.

Similarly, as tremor induced by Parkinson's disease decreases, processor 210, via the one or more of electrodes 116, 118, detects a decrease in the magnitude of the neurological signals within the Beta frequency band of patient 112. In another example, the signal is a neurological signal within the Gamma frequency band of brain 120 of patient 112. The signal within the Gamma frequency band of patient 112 may also correlate to one or more side effects of the electrical stimulation therapy. However, in contrast to neurological signals within the Beta frequency band, generally speaking, neurological signals within the Gamma frequency band of patient 112 may be approximately inversely proportional to the severity of the side effects of the electrical stimulation therapy. For example, as side effects due to electrical stimulation therapy increase, processor 210, via the one or more of electrodes 116, 118, detects a decrease in the magnitude of the signal within the Gamma frequency band of patient 112. Similarly, as side effects due to electrical stimulation therapy decrease, processor 210, via the one or more of electrodes 116, 118, detects an increase in the magnitude of the signal within the Gamma frequency band of patient 112.

In some examples, processor 210 continuously measures the signal from any signal modality in real time. In other examples, processor 210 periodically samples the signal according to a predetermined frequency or after a predetermined amount of time. In some examples, processor 210 periodically samples the signal at a frequency of approximately 150 Hertz.

Furthermore, processor 210 delivers electrical stimulation therapy that is constrained by an upper limit and a lower limit of a therapeutic window. In some examples, values defining the therapeutic window are stored within memory 211 of IMD 106. For example, in response to detecting that the brain signal has deviated from the homeostatic window, processor 210 of IMD 106 may adjust one or more parameters of the electrical stimulation therapy to provide responsive treatment to patient 112. For example, in response to detecting that the signal has exceeded an upper threshold of the homeostatic window and prior to delivering the electrical stimulation therapy, processor 210 increases an amplitude of stimulation (e.g., but not above the upper limit) in order to bring the signal back down below the upper threshold. For example, in a voltage-controlled system wherein the clinician has set the upper limit of the therapeutic window to be 3 Volts, processor 210 can increase the voltage amplitude to values no greater than 3 Volts in an attempt to decrease the brain signal below the upper threshold.

In another example, in response to detecting that the signal has fallen below a lower threshold of the homeostatic window and prior to delivering the electrical stimulation therapy, processor 210 decreases the voltage amplitude, for example, but not lower than the magnitude of the lower limit. For example, in the above voltage-controlled system wherein the clinician has set the lower bound of the therapeutic window to be 1.2 Volts, processor 210 can decrease the voltage amplitude down to no lower than 1.2 Volts in an attempt to raise the brain signal back above the lower threshold and into the homeostatic window. Thus, processor 210 of IMD 106 may deliver adaptive DBS to patient 112 wherein the one or more parameters defining the adaptive DBS is within the therapeutic window defined by a lower and upper limit for the parameter.

In another example, processor 210, via telemetry module 208 and from external programmer 104, receives instructions to adjust one or more limits of the therapeutic window. For example, such instructions may be in response to patient feedback on the efficacy of the electrical stimulation therapy, or in response to one or more sensors that have detected a signal of the patient. Such signals from sensors may include neurological signals, such as a signal within the Beta frequency band or signal within the Gamma frequency band of brain 120 of patient 112, or physiological parameters and measurements, such as a signal indicating one or more of a patient activity level, posture, and respiratory function. Further, such signals from sensors may indicate a lack of reduction of one or more symptoms of the patient 112, such as tremor or rigidity or the presence of side effects due to electrical stimulation therapy, such as paresthesia. In response to these instructions, processor 210 may adjust one or more thresholds of the homeostatic window. For example, processor 210 may adjust the magnitude of the upper threshold, the lower threshold, or shift the overall position of the homeostatic window such that the threshold, defined by the homeostatic window, for adjustment of the one or more parameters of electrical stimulation, is itself adjusted. Thereafter, processor 210, via electrodes 116 and 118, delivers the adjusted electrical stimulation to patient 112.

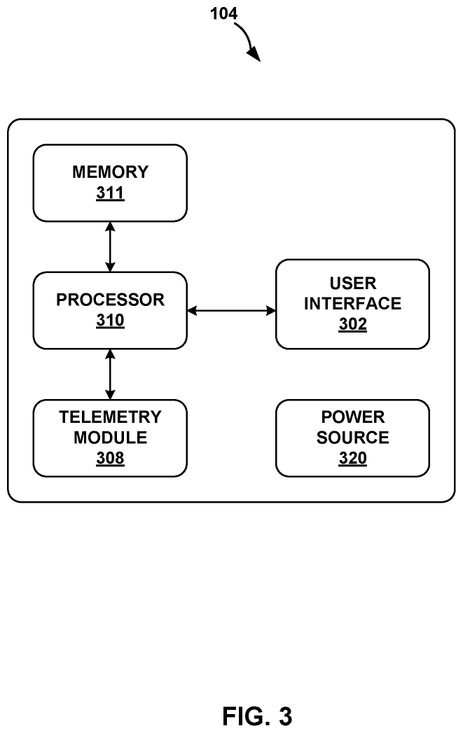

FIG. 3 is a block diagram of the external programmer 104 of FIG. 1. Although programmer 104 may generally be described as a hand-held device, programmer 104 may be a larger portable device or a more stationary device. In some examples, programmer 104 may be referred to as a tablet computing device. In addition, in other examples, programmer 104 may be included as part of an external charging device or include the functionality of an external charging device. As illustrated in FIG. 3, programmer 104 may include a processor 310, memory 311, user interface 302, telemetry module 308, and power source 320. Memory 311 may store instructions that, when executed by processor 310, cause processor 310 and external programmer 104 to provide the functionality ascribed to external programmer 104 throughout this disclosure. Each of these components, or modules, may include electrical circuitry that is configured to perform some or all of the functionality described herein. For example, processor 310 may include processing circuitry configured to perform the processes discussed with respect to processor 310.

In general, programmer 104 comprises any suitable arrangement of hardware, alone or in combination with software and/or firmware, to perform the techniques attributed to programmer 104, and processor 310, user interface 302, and telemetry module 308 of programmer 104. In various examples, programmer 104 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. Programmer 104 also, in various examples, may include a memory 311, such as RAM, ROM, PROM, EPROM, EEPROM, flash memory, a hard disk, a CD-ROM, comprising executable instructions for causing the one or more processors to perform the actions attributed to them. Moreover, although processor 310 and telemetry module 308 are described as separate modules, in some examples, processor 310 and telemetry module 308 may be functionally integrated with one another. In some examples, processor 310 and telemetry module 308 correspond to individual hardware units, such as ASICs, DSPs, FPGAs, or other hardware units.

Memory 311 (e.g., a storage device) may store instructions that, when executed by processor 310, cause processor 310 and programmer 104 to provide the functionality ascribed to programmer 104 throughout this disclosure. For example, memory 311 may include instructions that cause processor 310 to obtain a parameter set from memory, select a spatial electrode movement pattern, or receive a user input and send a corresponding command to IMD 104, or instructions for any other functionality. In addition, memory 311 may include a plurality of programs, where each program includes a parameter set that defines stimulation therapy.

User interface 302 may include a button or keypad, lights, a speaker for voice commands, a display, such as a liquid crystal (LCD), light-emitting diode (LED), or organic light-emitting diode (OLED). In some examples the display may be a touch screen. User interface 302 may be configured to display any information related to the delivery of stimulation therapy, identified patient behaviors, sensed patient parameter values, patient behavior criteria, or any other such information. User interface 302 may also receive user input via user interface 302. The input may be, for example, in the form of pressing a button on a keypad or selecting an icon from a touch screen. User interface 302 may provide selectable options to set up sensing modality configurations, assign a sensing modality configuration to specific therapy modes or patient states, or otherwise select or manage the sensing modality configurations and switching described herein. User interface 302 may refer to hardware configured to present information to a user and/or receive input from the user. In some examples, processor 310 directly controls this hardware. In other examples, processor 310 may communicate with drive hardware that controls hardware of user interface 302. In some example, user interface 302 may include display and/or interactive display configurations as described herein.

Telemetry module 308 may support wireless communication between IMD 106 and programmer 104 under the control of processor 310. Telemetry module 308 may also be configured to communicate with another computing device via wireless communication techniques, or direct communication through a wired connection. In some examples, telemetry module 308 provides wireless communication via an RF or proximal inductive medium. In some examples, telemetry module 308 includes an antenna, which may take on a variety of forms, such as an internal or external antenna.

Examples of local wireless communication techniques that may be employed to facilitate communication between programmer 104 and IMD 106 include RF communication according to the 802.11 or Bluetooth specification sets or other standard or proprietary telemetry protocols. In this manner, other external devices may be capable of communicating with programmer 104 without needing to establish a secure wireless connection. As described herein, telemetry module 308 may be configured to transmit a spatial electrode movement pattern or other stimulation parameter values to IMD 106 for delivery of stimulation therapy.

According to the techniques of the disclosure, in some examples, processor 310 of external programmer 104 defines the parameters of a homeostatic therapeutic window, stored in memory 311, for delivering adaptive DBS to patient 112. In one example, processor 311 of external programmer 104, via telemetry module 308, issues commands to IMD 106 causing IMD 106 to deliver electrical stimulation therapy via electrodes 116, 118 via leads 114.

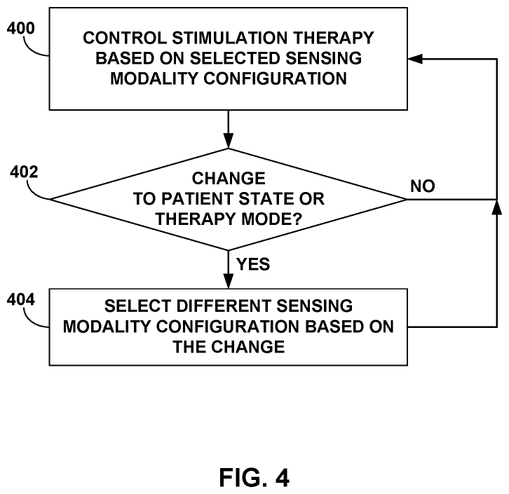

FIG. 4 is a flowchart illustrating an example technique for switching between different sensing modality configurations. The example of FIG. 4 will be described as being performed by processor 210 of IMD 106, but other processing circuitry, devices, or combinations thereof, may perform this process in other examples.

In the example if FIG. 4, processor 210 controls stimulation therapy based on a selected sensing modality configuration (400). The selected sensing modality configuration may include one or more different sensing modalities, and processor 210 may adjust a parameter that defines the stimulation therapy (e.g., an amplitude of electrical stimulation) based on the outputs from the sensing modalities of the sensing modality configuration. If processor 210 does not detect a change to the patient state or therapy mode (“NO” branch of block 402), processor 210 may continue to control the stimulation therapy using the same sensing modality configuration (400). Different patient states may include different patient activity levels, posture states, sleep states, disease states, etc. Different therapy modes may include stimulation programs that have different electrode configurations, different pulse frequencies, different stimulation cycling plans, different combinations of stimulation signals, the addition or removal of medication, etc.

However, if processor 210 detects a change to the patient state or therapy mode (“YES” branch of block 402), processor 210 the selects a different sensing modality configuration based on the detected change (404). For example, the detected patient state or therapy mode may be associated with a specific sensing modality configuration, and processor 210 may select that associated sensing modality configuration and switch to that new sensing modality configuration for continuing to control stimulation therapy (400). This process can be used for any sensing modalities or combinations thereof.

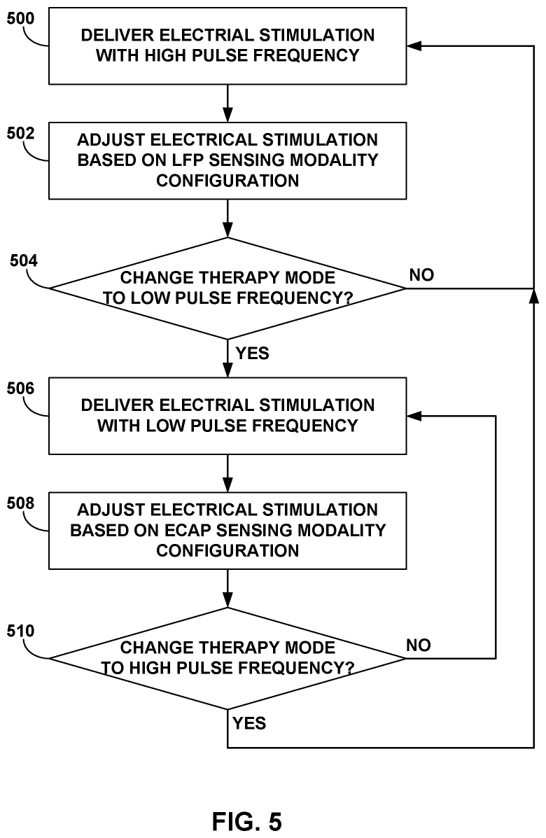

FIG. 5 is a flowchart illustrating an example technique for switching between different sensing modality configurations for different pulse frequencies of electrical stimulation. The example of FIG. 5 will be described as being performed by processor 210 of IMD 106, but other processing circuitry, devices, or combinations thereof, may perform this process in other examples.

In the example of FIG. 5, processor 210 controls stimulation circuitry to deliver electrical stimulation with a high pulse frequency (500). A high pulse frequency may be a pulse frequency that is at or above a threshold frequency above which a certain sensing modality may not be used. In this example, this sensing modality that may not be used high above threshold frequencies may be ECAP signal detection. For high frequency pulses, the ECAP signal may be covered up by one or more pulses that are delivered before the end of the ECAP signal. This overlap may render the ECAP signal unusable for feedback purposes. However, other sensing modalities may still be viable feedback options for adjusting therapy during high pulse frequencies, such as LFP sensing. Therefore, processor 210 may adjust the electrical stimulation (e.g., one or more parameter values that define the stimulation) based on the LFP sensing modality configuration (502). In some examples, the therapy mode may include stimulation at a high pulse frequency at one electrode combination that is delivered in combination with stimulation at a low pulse frequency at a different electrode combination. However, the high pulse frequency may still interfere with ECAP sensing associated with the low pulse frequency stimulation. If no change occurs to a low pulse frequency therapy mode (“NO” branch of block 504), processor 210 may continue to adjust stimulation based on the LFP sensing modality.

If processor 210 changes the therapy mode to a lower pulse frequency below the threshold frequency (“YES” branch of block 504), processor 210 delivers electrical stimulation with the low pulse frequency (506) and adjust one or more parameters defining the electrical stimulation based on an ECAP sensing modality configuration that may be appropriate for feedback during the lower pulse frequency stimulation (508). If no change occurs to the high pulse frequency therapy mode (“NO” branch of block 510), processor 210 may continue to adjust stimulation based on the ECAP sensing modality. If processor 210 changes the therapy mode to the high pulse frequency again (“YES” branch of block 510), processor 210 delivers electrical stimulation with the high pulse frequency (500) and controls stimulation via the LFP signals again.

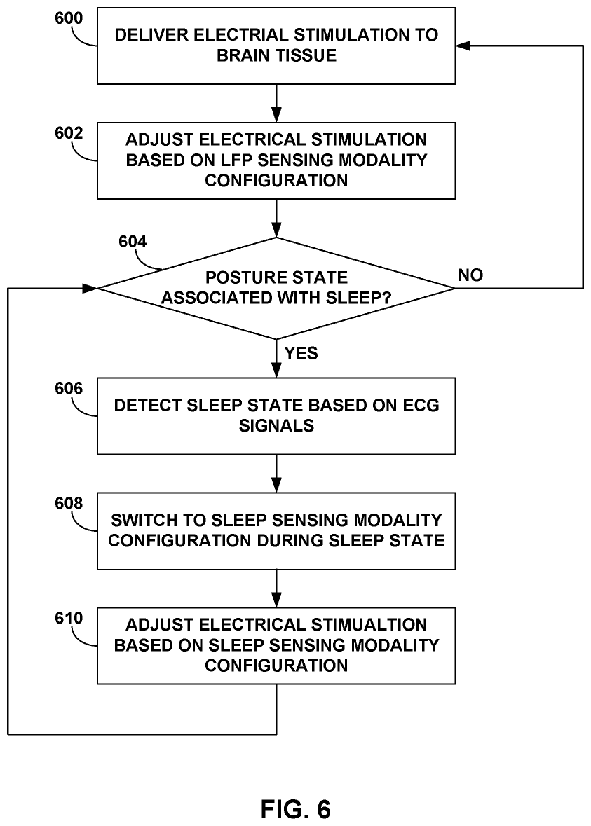

FIG. 6 is a flowchart illustrating an example technique for switching between different sensing modality configurations in different posture states. The example of FIG. 6 will be described as being performed by processor 210 of IMD 106, but other processing circuitry, devices, or combinations thereof, may perform this process in other examples.

In the example of FIG. 6, processor 210 controls stimulation circuitry, such as stimulation generator 202, to deliver electrical stimulation therapy to brain tissue of the patient (600). Processor 210 can then adjust one or more parameters defining the electrical stimulation based on an LFP sensing modality configuration (602). Processor 210 may then monitor, using a sensing modality such as an accelerometer signal, a posture state of the patient that may be associated with sleep. When the patient is awake, processor 210 may control delivery of stimulation to reduce symptoms of the patient. However, when the patient is asleep, the system may switch to a different therapy mode and/or a different sensing mode.

As long as processor 210 determines that the posture state is not associated with sleep (“NO” branch of block 604), processor 210 continues to control delivery of the stimulation based on LFP signals (602). If processor 210 determines that the posture state, such as an acceleration signal indicative of patient sleep, is associated with sleep (“YES” branch of block 604), processor 210 may begin a sensing modality to detect the sleep state (606). In this manner, the accelerometer sensing modality may be a trigger for another sensing modality for determining the sleep state of the patient. This sleep state sensing modality may include another type of sensing, such as detecting the sleep state based on ECG signals for the patient. These ECG signals related to cardiac activity may be reflective of the sleep state of the patient. In some examples, processor 210 may control the sensing circuitry used to detect LFP signal to detect ECG signals instead.

Processor 210 can the switch to a sleep sending modality configuration during this sleep state (608). The sleep sensing modality configuration may include the ECG signals and/or another type of sensing modality. Processor 210 can then adjust electrical stimulation based on the signal from the sleep sensing modality configuration (610). For example, processor 210 may cycle stimulation on and off to minimize stimulation usage, adjust upper or lower thresholds, or completely stop electrical stimulation. Processor 210 may adjust stimulation usage in this manner to reduce power consumption when the patient would not be affected by normal symptoms and/or resume stimulation to suppress symptoms when the patient is sleeping lightly or may become away in order to facilitate the patient falling back asleep again. If the posture state is no longer associated with sleep (“NO” branch of block 604), processor 210 may resume normal awake stimulation (600) and sensing modality configuration (602).

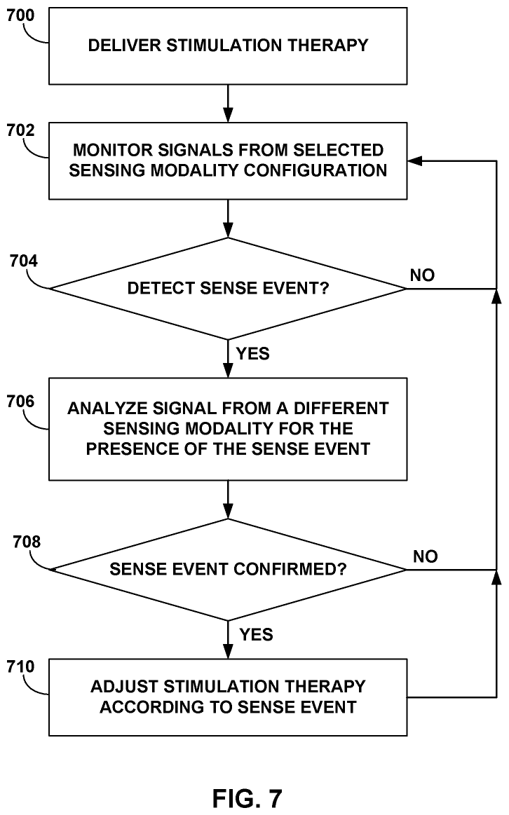

FIG. 7 is a flowchart illustrating an example technique for confirming sense events with a different sensing modality configuration. The example of FIG. 7 will be described as being performed by processor 210 of IMD 106, but other processing circuitry, devices, or combinations thereof, may perform this process in other examples.

In the example of FIG. 7, processor 210 controls stimulation circuitry, such as stimulation generator 202, to deliver electrical stimulation therapy to the patient (700). Processor 210 can then monitor signals from a selected sensing modality configuration (702). These signals may be from one sensing modality or two or more sensing modalities that may be configured to sense an event. The sense event may be the signal exceeding a threshold, being within a range for a certain period of time, or any other indication of patient status. If processor 210 does not detect a sense event from the sensing modality configuration (“NO” branch of block 704), processor 210 continues to monitor the signals from the sensing modality configuration (702).

If processor 210 detects the sense event from the sensing modality configuration (“YES” branch of block 704), processor 210 analyzes a signal from a different sensing modality for the presence of the sense event (706). In some examples, the different sensing modality signal may be sensed and stored for the same time period in which the sense event was initially detected. In this manner, the signal may be stored in a buffer or other memory as needed for processor 210 to look back at that signal for confirmation. In other examples, processor 210 may begin sensing with the different sensing modality in response to detecting the sense event such that the different sensing modality is sensing at a different time than the time in which the initial sense event was detected. In any case, if processor 210 confirms the presence of the sense event detected by the sensing modality configuration with the different sensing modality (“YES” branch of block 708), processor 210 adjusts one or more parameters defining stimulation therapy according to the sense event (710).

If processor 210 does not confirm the presence of the sense event detected by the sensing modality configuration with the different sensing modality (“NO” branch of block 708), processor 210 does not adjust stimulation based on the sense event and continues to monitor signals from the sensing modality configuration (702). In some examples, processor 210 may store data indicating when the sensing modality configuration detects the sense event and whether or not the different sensing modality confirmed the presence of the sense event. In this manner, processor 210 can automatically adjust the sensing modality configuration if unconfirmed sense events happen frequently or deliver an alert to a user that the sensing modality confirmation may need to be changed to more accurately detect sense events for improved closed-loop control of therapy.

The process of FIG. 7 may applied to any sensing modality described herein. That is, processor 210 may be configured to confirm signals or sense events from one, some, or all sensing modalities with a different sensing modality. Processor 210 may have instructions to perform this confirmation for various sensing modalities that may be prone to possible error or trigger confirmation in response to detecting anomalies or artifacts with the sensed signal.

The following examples are described herein. Example 1. A system comprising: a memory configured to store a plurality of different sensing modality configurations, each sensing modality configuration of the plurality of sensing modality configurations defining at least one sensing modality of a plurality of sensing modalities; processing circuitry configured to: control stimulation generation circuitry to deliver electrical stimulation therapy based on a first sensing modality configuration of the plurality of different sensing modality configurations; determine a change to at least one of a patient state or a therapy mode; responsive to determining the change, select a second sensing modality configuration of the plurality of different sensing modality configurations; and control the stimulation generation circuitry to deliver the electrical stimulation therapy based on the second sensing modality configuration instead of the first sensing modality configuration.

Example 2. The system of example 1, wherein the plurality of sensing modalities comprise at least one of a biochemical sensing modality, an electrophysical sensing modality, a patient motion sensing modality, or a posture state sensing modality.

Example 3. The system of example 2, wherein the plurality of sensing modalities comprises the biochemical sensing modality, the biochemical sensing modality configured to sense a presence of one or more neurotransmitters in a patient.

Example 4. The system of example 2, wherein the plurality of sensing modalities comprises the electrophysical sensing modality, the electrophysical sensing modality configured to sense at least one of a local field potential sensing modality or an evoked neural response sensing modality.

Example 5. The system of example 2, wherein the plurality of sensing modalities comprises an accelerometer sensing modality, the accelerometer sensing modality configured to sense at least one of an activity or a posture of the patient.

Example 6. The system of any of examples 1 through 5, wherein at least one of the first sensing modality configuration or the second sensing modality configuration comprises at least three sensing modalities, and wherein the processing circuitry is configured to control the electrical stimulation based on a subset of at least the two sensing modalities of the plurality of sensing modalities.

Example 7. The system of any of examples 1 through 6, wherein the patient state comprises a disease state.

Example 8. The system of any of examples 1 through 7, wherein the patient state comprises one patient state of a plurality of patient states, and wherein one or more sensing modalities of the plurality of sensing modalities are assigned to be used with respective patient states of the plurality of patient states

Example 9. The system of any of examples 1 through 7, wherein the therapy mode comprises one therapy mode of a plurality of therapy modes configured to provide different respective therapies to a patient.

Example 10. The system of example 9, wherein the plurality of therapy modes define different respective stimulation pulse frequencies of the electrical stimulation therapy.

Example 11. The system of example 9, wherein one or more sensing modalities of the plurality of sensing modalities are assigned to be used with respective therapy modes of the plurality of therapy modes.

Example 12. The system of example 11, wherein the processing circuitry is configured to: control sensing circuitry to sense local field potentials (LFPs) as one sensing modality of the plurality of sensing modalities during one or more first therapy modes that include electrical stimulation therapy using first stimulation pulse frequencies greater than a threshold frequency for sensing evoked compound action potential (ECAP) signals; and control the sensing circuitry to sense ECAP signals as another sensing modality of the plurality of sensing modalities during one or more second therapy modes that include electrical stimulation therapy using second stimulation pulse frequencies less than the threshold frequency for sensing the ECAP signals.

Example 13. The system of any of examples 1 through 12, wherein the processing circuitry is configured to: determine an error associated with a current sensing modality of the plurality of sensing modalities; responsive to determining the error, switch from the current sensing to a different sensing modality of the plurality of sensing modalities; and control sensing according to the different sensing modality.

Example 14. The system of any of examples 1 through 13, wherein the processing circuitry is configured to control stimulation generation circuitry to deliver electrical stimulation therapy based on the first sensing modality by at least: detecting a first sense event using the first sensing modality; confirming presence of the first sense event based on a third sensing modality different than the first sensing modality; and responsive to confirming the presence of the first sense event, adjust a parameter that at least partially defines the electrical stimulation therapy.

Example 15. The system of any of examples 1 through 14, further comprising the stimulation generation circuitry.

Example 16. The system of any of examples 1 through 15, further comprising an implantable medical device comprising the memory and the processing circuitry.

Example 17. A method comprising: storing, by a memory, a plurality of different sensing modality configurations, each sensing modality configuration of the plurality of sensing modality configurations defining at least one sensing modality of a plurality of sensing modalities; controlling, by processing circuitry, stimulation generation circuitry to deliver electrical stimulation therapy based on a first sensing modality configuration of the plurality of different sensing modality configurations; determining, by processing circuitry, a change to at least one of a patient state or a therapy mode; responsive to determining the change, selecting, by processing circuitry, a second sensing modality configuration of the plurality of different sensing modality configurations; and controlling, by processing circuitry, the stimulation generation circuitry to deliver the electrical stimulation therapy based on the second sensing modality configuration instead of the first sensing modality configuration.

Example 18. The system of example 17, wherein the plurality of sensing modalities comprise at least one of a biochemical sensing modality, an electrophysical sensing modality, a patient motion sensing modality, or a posture state sensing modality.

Example 19. The system of example 18, wherein the plurality of sensing modalities comprises the biochemical sensing modality, the biochemical sensing modality configured to sense a presence of one or more neurotransmitters in a patient.

Example 20. The system of example 18, wherein the plurality of sensing modalities comprises the electrophysical sensing modality, the electrophysical sensing modality configured to sense at least one of a local field potential sensing modality or an evoked neural response sensing modality.

Example 21. The system of example 18, wherein the plurality of sensing modalities comprises an accelerometer sensing modality, the accelerometer sensing modality configured to sense at least one of an activity or a posture of the patient.

Example 22. The system of any of examples 17 through 21, wherein at least one of the first sensing modality configuration or the second sensing modality configuration comprises at least three sensing modalities, and wherein the processing circuitry is configured to control the electrical stimulation based on a subset of at least the two sensing modalities of the plurality of sensing modalities.

Example 23. The system of any of examples 17 through 22, wherein the patient state comprises a disease state.

Example 24. The system of any of examples 17 through 23, wherein the patient state comprises one patient state of a plurality of patient states, and wherein one or more sensing modalities of the plurality of sensing modalities are assigned to be used with respective patient states of the plurality of patient states.

Example 25. The system of any of examples 1 through 24, wherein the therapy mode comprises one therapy mode of a plurality of therapy modes configured to provide different respective therapies to a patient.

Example 26. The system of example 25, wherein the plurality of therapy modes define different respective stimulation pulse frequencies of the electrical stimulation therapy.

Example 27. The system of example 25, wherein one or more sensing modalities of the plurality of sensing modalities are assigned to be used with respective therapy modes of the plurality of therapy modes.

Example 28. The system of example 27, wherein the processing circuitry is configured to: control sensing circuitry to sense local field potentials (LFPs) as one sensing modality of the plurality of sensing modalities during one or more first therapy modes that include electrical stimulation therapy using first stimulation pulse frequencies greater than a threshold frequency for sensing evoked compound action potential (ECAP) signals; and control the sensing circuitry to sense ECAP signals as another sensing modality of the plurality of sensing modalities during one or more second therapy modes that include electrical stimulation therapy using second stimulation pulse frequencies less than the threshold frequency for sensing the ECAP signals.

Example 29. The system of any of examples 17 through 28, wherein the processing circuitry is configured to: determine an error associated with a current sensing modality of the plurality of sensing modalities; responsive to determining the error, switch from the current sensing to a different sensing modality of the plurality of sensing modalities; and control sensing according to the different sensing modality.

Example 30. The system of any of examples 17 through 29, wherein the processing circuitry is configured to control stimulation generation circuitry to deliver electrical stimulation therapy based on the first sensing modality by at least: detecting a first sense event using the first sensing modality; confirming presence of the first sense event based on a third sensing modality different than the first sensing modality; and responsive to confirming the presence of the first sense event, adjust a parameter that at least partially defines the electrical stimulation therapy.

Example 31. A non-transitory computer-readable medium comprising instructions that, when executed, control processing circuitry to: store, in a memory, a plurality of different sensing modality configurations, each sensing modality configuration of the plurality of sensing modality configurations defining at least one sensing modality of a plurality of sensing modalities; control stimulation generation circuitry to deliver electrical stimulation therapy based on a first sensing modality configuration of the plurality of different sensing modality configurations; determine a change to at least one of a patient state or a therapy mode; responsive to determining the change, select a second sensing modality configuration of the plurality of different sensing modality configurations; and control the stimulation generation circuitry to deliver the electrical stimulation therapy based on the second sensing modality configuration instead of the first sensing modality configuration.

The techniques described in this disclosure may be implemented, at least in part, in hardware, software, firmware or any combination thereof. For example, various aspects of the described techniques may be implemented within one or more processors, such as fixed function processing circuitry and/or programmable processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A system comprising:
a memory configured to store a plurality of different sensing modality configurations, each sensing modality configuration of the plurality of sensing modality configurations defining at least one sensing modality of a plurality of sensing modalities;
processing circuitry configured to:
control stimulation generation circuitry to deliver electrical stimulation therapy based on a first sensing modality configuration of the plurality of different sensing modality configurations;
determine a change to at least one of a patient state or a therapy mode;
responsive to determining the change, select a second sensing modality configuration of the plurality of different sensing modality configurations; and
control the stimulation generation circuitry to deliver the electrical stimulation therapy based on the second sensing modality configuration instead of the first sensing modality configuration.

2. The system of claim 1, wherein the plurality of sensing modalities comprise at least one of a biochemical sensing modality, an electrophysical sensing modality, a patient motion sensing modality, or a posture state sensing modality.

3. The system of claim 2, wherein the plurality of sensing modalities comprises the biochemical sensing modality, the biochemical sensing modality configured to sense a presence of one or more neurotransmitters in a patient.

4. The system of claim 2, wherein the plurality of sensing modalities comprises the electrophysical sensing modality, the electrophysical sensing modality configured to sense at least one of a local field potential sensing modality or an evoked neural response sensing modality.

5. (canceled)

6. The system of claim 1, wherein at least one of the first sensing modality configuration or the second sensing modality configuration comprises at least three sensing modalities, and wherein the processing circuitry is configured to control the electrical stimulation based on a subset of at least the two sensing modalities of the plurality of sensing modalities.

7. (canceled)

8. The system of claim 1, wherein the patient state comprises one patient state of a plurality of patient states, and wherein one or more sensing modalities of the plurality of sensing modalities are assigned to be used with respective patient states of the plurality of patient states.

9. The system of claim 1, wherein the therapy mode comprises one therapy mode of a plurality of therapy modes configured to provide different respective therapies to a patient, wherein
each of the plurality of therapy modes defines different respective stimulation pulse frequencies of the electrical stimulation therapy; and
the plurality of therapy modes define different respective stimulation pulse frequencies of the electrical stimulation therapy.

10-11. (canceled)

12. The system of claim 9, wherein the processing circuitry is configured to:
control sensing circuitry to sense local field potentials (LFPs) as one sensing modality of the plurality of sensing modalities during one or more first therapy modes that include electrical stimulation therapy using first stimulation pulse frequencies greater than a threshold frequency for sensing evoked compound action potential (ECAP) signals; and
control the sensing circuitry to sense ECAP signals as another sensing modality of the plurality of sensing modalities during one or more second therapy modes that include electrical stimulation therapy using second stimulation pulse frequencies less than the threshold frequency for sensing the ECAP signals.

13. The system of claim 1, wherein the processing circuitry is configured to:
determine an error associated with a current sensing modality of the plurality of sensing modalities;
responsive to determining the error, switch from the current sensing to a different sensing modality of the plurality of sensing modalities; and
control sensing according to the different sensing modality.

14. The system of claim 1, wherein the processing circuitry is configured to control stimulation generation circuitry to deliver electrical stimulation therapy based on the first sensing modality by at least:
detecting a first sense event using the first sensing modality;
confirming presence of the first sense event based on a third sensing modality different than the first sensing modality; and
responsive to confirming the presence of the first sense event, adjust a parameter that at least partially defines the electrical stimulation therapy.

15. (canceled)

16. A method comprising:
storing, by a memory, a plurality of different sensing modality configurations, each sensing modality configuration of the plurality of sensing modality configurations defining at least one sensing modality of a plurality of sensing modalities;
controlling, by processing circuitry, stimulation generation circuitry to deliver electrical stimulation therapy based on a first sensing modality configuration of the plurality of different sensing modality configurations;
determining, by processing circuitry, a change to at least one of a patient state or a therapy mode;
responsive to determining the change, selecting, by processing circuitry, a second sensing modality configuration of the plurality of different sensing modality configurations; and
controlling, by processing circuitry, the stimulation generation circuitry to deliver the electrical stimulation therapy based on the second sensing modality configuration instead of the first sensing modality configuration.

17. The method of claim 16, wherein the plurality of sensing modalities comprise at least one of a biochemical sensing modality, an electrophysical sensing modality, a patient motion sensing modality, or a posture state sensing modality.

18. The method of claim 17, wherein the plurality of sensing modalities comprises the biochemical sensing modality, the biochemical sensing modality configured to sense a presence of one or more neurotransmitters in a patient.

19. The method of claim 17, wherein the plurality of sensing modalities comprises the electrophysical sensing modality, the electrophysical sensing modality configured to sense at least one of a local field potential sensing modality or an evoked neural response sensing modality.

20. The method of claim 16, wherein at least one of the first sensing modality configuration or the second sensing modality configuration comprises at least three sensing modalities, and wherein the processing circuitry is configured to control the electrical stimulation based on a subset of at least the two sensing modalities of the plurality of sensing modalities.

21. The method of claim 16, wherein the patient state comprises one patient state of a plurality of patient states, and wherein one or more sensing modalities of the plurality of sensing modalities are assigned to be used with respective patient states of the plurality of patient states.

22. The method of claim 16, wherein:
the therapy mode comprises one therapy mode of a plurality of therapy modes configured to provide different respective therapies to a patient;
the plurality of therapy modes define different respective stimulation pulse frequencies of the electrical stimulation therapy; and
or more sensing modalities of the plurality of sensing modalities are assigned to be used with respective therapy modes of the plurality of therapy modes

23. The method of claim 23, wherein the processing circuitry is configured to:
control sensing circuitry to sense local field potentials (LFPs) as one sensing modality of the plurality of sensing modalities during one or more first therapy modes that include electrical stimulation therapy using first stimulation pulse frequencies greater than a threshold frequency for sensing evoked compound action potential (ECAP) signals; and
control the sensing circuitry to sense ECAP signals as another sensing modality of the plurality of sensing modalities during one or more second therapy modes that include electrical stimulation therapy using second stimulation pulse frequencies less than the threshold frequency for sensing the ECAP signals.

24. The method of claim 16, wherein the processing circuitry is configured to:
determine an error associated with a current sensing modality of the plurality of sensing modalities;
responsive to determining the error, switch from the current sensing to a different sensing modality of the plurality of sensing modalities; and
control sensing according to the different sensing modality.

25. The method of claim 16, wherein the processing circuitry is configured to control stimulation generation circuitry to deliver electrical stimulation therapy based on the first sensing modality by at least:
detecting a first sense event using the first sensing modality;
confirming presence of the first sense event based on a third sensing modality different than the first sensing modality; and
responsive to confirming the presence of the first sense event, adjust a parameter that at least partially defines the electrical stimulation therapy.
